(19) United States
(12) Patent Application Publication
(10) Pub. No.: US 2024/0269326 A1
(43) Pub. Date: Aug. 15, 2024

(54) METHODS AND COMPOSITIONS FOR TREATING OCULAR DISEASES AND DISORDERS

(71) Applicant: Abeona Therapeutics Inc., New York, NY (US)

(72) Inventors: Linas PADEGIMAS, Mayfield, OH (US); Brian KEVANY, New York, NY (US)

(21) Appl. No.: 18/561,784

(22) PCT Filed: May 18, 2022
(86) PCT No.: PCT/US2022/029797

§ 371 Date: Nov. 17, 2023

Related U.S. Application Data
(60) Provisional application No. 63/189,836, filed on May 18, 2021.
(60) Provisional application No. 63/275,527, filed on Nov. 4, 2021.
(60) Provisional application No. 63/334,949, filed on Apr. 26, 2022.

(51) Int. Cl. A61K 48/00; A61K 9/00; A61K 38/17; A61K 38/46; A61P 27/02; C07K 14/005; C12N 15/113; C12N 15/86

(52) U.S. Cl. CPC A61K 48/005; A61K 9/0048; A61K 38/1709; A61K 38/46; A61K 48/0075; A61P 27/02; C07K 14/005; C12N 15/113; C12N 15/86; C12Y 306/05005; C12N 2750/14122; C12N 2750/14143

(57) ABSTRACT

Provided herein are recombinant AAV vectors, AAV viral vectors, capsid proteins, and administration methods for improved gene therapy, and methods for their manufacture and use.

### CROSS-REFERENCE TO RELATED APPLICATIONS

This application claims the benefit of U.S. Provisional Application No. 63/189,836, filed May 18, 2021, U.S. Provisional Application No. 63/275,527, filed Nov. 4, 2021, and U.S. Provisional Application No. 63/334,949, filed Apr. 26, 2022, all of which are herein incorporated by reference in their entireties.

### INCORPORATION BY REFERENCE OF SEQUENCE LISTING

The contents of the text file submitted electronically herewith are incorporated herein by reference in their entirety: A computer readable format copy of the Sequence Listing (filename: ABEO_008_03WO_SeqList_ST25.TXT, date created: May 17, 2022, file size: about 636 kb).

### BACKGROUND

Adeno-associated viral vectors are promising delivery vectors for gene therapy. However, their therapeutic efficacy is undermined by the vectors' delivery route, delivery efficiency. Therefore, there is an urgent need for new strategies for delivering selected AAV viral vectors with a better therapeutic potential.

### SUMMARY

The present disclosure relates generally to the field of gene therapy and in particular, to recombinant adeno-associated viral (AAV) vector particles (also known as AAV viral vectors) with novel capsid proteins, their manufacture, their delivery methods, and their use to deliver transgenes to treat or prevent a disease or disorder.

In one aspect, the disclosure provides methods of treating an ophthalmic disease or disorder in a subject in need thereof, comprising para-retinal administration of an AAV viral vector to the subject, wherein the AAV viral vector comprises an AAV capsid protein comprising the amino acid sequence of SEQ ID NO: 2.

In one aspect, the disclosure provides methods of treating an ophthalmic disease or disorder in a subject in need thereof, comprising para-retinal administration of an AAV viral vector to the subject, wherein the AAV viral vector comprises an AAV capsid protein comprising or consisting of an amino acid sequence that is at least 95%, at least 98%, at least 99%, at least 99.5%, or 100% identical to any one of SEQ ID NO: 1-3, 30-34, 49, 67, 84, and 164.

In embodiments, the AAV viral vector comprises an AAV capsid protein comprising an amino acid sequence that is up to 1, 2, 3, 4, 5, 6, 7, 8, 9, or 10 amino acids different from any one of SEQ ID NO: 1-3, 30-34, 49, 67, 84 and 164. In embodiments, the AAV viral vector comprises an AAV capsid protein comprising or consisting of the amino acid sequence of SED ID NO: 2 or an amino acid sequence that is up to 1, 2, 3, 4, 5, 6, 7, 8, 9, or 10 amino acids different from SEQ ID NO: 2. In embodiments, the AAV capsid protein comprises a leucine (L) at amino acid 129 of SEQ ID NO: 2, an asparagine (N) at amino acid 586 of SEQ ID NO: 2, and a glutamic acid (E) at amino acid 723 of SEQ ID NO: 2. In embodiments, the AAV viral vector comprises an AAV capsid protein comprising or consisting of the amino acid sequence of SED ID NO: 1 or an amino acid sequence that is up to 2, 2, 3, 4, 5, 6, 7, 8, 9, or 10 amino acids different from SEQ ID NO: 1. In embodiments, the AAV capsid protein comprises a leucine (L) at amino acid 129, a proline (P) at amino acid 148, a arginine (R) at amino acid 152, a serine (S) at amino acid 153, a threonine (T) at amino acid 158, a lysine (K) at amino acid 163, a arginine (R) at amino acid 169, a tryptophan (W) at amino acid 306, a phenylalanine (F) at amino acid 308, and a asparagine (N) at amino acid 319, wherein the amino acid positions are numbered with respect to SEQ ID NO: 1. In embodiments, the AAV viral vector comprises an AAV capsid protein comprising or consisting of the amino acid sequence of SED ID NO: 164 or an amino acid sequence that is up to 1, 2, 3, 4, 5, 6, 7, 8, 9, or 10 amino acids different from SEQ ID NO: 164. In embodiments, the AAV viral vector comprises an AAV capsid protein comprising or consisting of the amino acid sequence of SED ID NO: 67 or an amino acid sequence that is up to 1, 2, 3, 4, 5, 6, 7, 8, 9, or 10 amino acids different from SEQ ID NO: 67. In embodiments, the AAV viral vector comprises an AAV capsid protein comprising or consisting of the amino acid sequence of SED ID NO: 3 or an amino acid sequence that is up to 2, 2, 3, 4, 5, 6, 7, 8, 9, or 10 amino acids different from SEQ ID NO: 3.

In embodiments, the AAV capsid protein comprises a VP3 portion comprising variable regions (VR) I to IX wherein:

(a) VR-II comprises amino acid sequence
(SEQ ID NO: 54)
DNNGVK,
(b) VR-III comprises amino acid sequence
(SEQ ID NO: 55)
NDGS,
(c) VR-IV comprises amino acid sequence
(SEQ ID NO: 56)
INGSGQNQQT
or
(SEQ ID NO: 171)
QSTGGTAGTQQ,
(d) VR-V comprises amino acid sequence
(SEQ ID NO: 57)
RVSTTTGQNNNSNFAWTA,
(e) VR-VI comprises amino acid sequence
(SEQ ID NO: 58)
HKEGEDRFFPLSG,
(f) VR-VII comprises amino acid sequence
(SEQ ID NO: 59)
KQNAARDNADYSDV,
(g) VR-VIII comprises amino acid sequence
(SEQ ID NO: 60)
ADNLQQQNTAPQI,
and
(h) VR-IX comprises amino acid sequence
(SEQ ID NO: 61)
NYYKSTSVDF.

In embodiments, the VR-I region comprises SASTGAS (SEQ ID NO. 52), NSTSGGSS (SEQ ID NO. 53), SSTSGGSS (SEQ ID NO. 87), or NGTSGGST (SEQ ID NO: 170).

In embodiments, wherein the ophthalmic disease or disorder is selected from the group consisting of dominant optic atrophy, retinitis pigmentosa, macular degeneration, an eye disorder related to mutations in the bestrophin-1 (BEST-1) gene, Leber congenital amaurosis, cone-rod dystrophy, Stargardt disease, choroideremia, Usher Syndrome, retinoschisis, Bietti's Crystalline Dystrophy and Achromatopsia. In embodiments, the retinitis pigmentosa is autosomal recessive, autosomal dominant, or X-linked. In embodiments, the eye disorder related to mutations in the BEST-1 gene is vitelliform macular dystrophy, age-related macular degeneration, autosomal dominant vitreoretinochoroidopathy, glaucoma, or cataract. In embodiments, the AAV viral vector comprises an AAV vector genome encoding a gene selected from SPATA7, LRAT, TULP1, AIPL1, RPGR, AIPL1, ABCA4, CHM, MY07A, CDH23, USH2A, CLRN1, RS1, CYP4V2, CNGA3, CNGB3, GNAT2, RHO, PDE6B, PDE6C, PDE6H, OPA1, OPA3, and BEST-1. In embodiments, the AAV viral vector comprises an AAV vector encoding an antisense RNA, microRNA, siRNA, or guide RNA (gRNA). In embodiments, the ophthalmic disease or disorder is related to a dysfunction of optic nerve.

In embodiments, the ophthalmic disease or disorder is Dominant Optic Atrophy. In embodiments, the AAV viral vector comprises an AAV vector genome comprising an OPA1 or OPA3 transgene.

In embodiments, the ophthalmic disease or disorder is Retinoschisis. In embodiments, the AAV viral vector comprises an AAV vector genome comprising a RS1 transgene.

In embodiments, the para-retinal administration comprises injecting at a distance of between 0-13 millimeters (mm), between 0-10 mm, between 0-5 mm, or between 0-3 mm, from the surface of the retina in the posterior vitreous cavity of the eye. In embodiments, the para-retinal administration comprises injecting at a distance of between 0-13 mm from the surface of the retina in the posterior vitreous cavity. In embodiments, the para-retinal administration comprises injecting at a distance of between 0-10 mm from the surface of the retina in the posterior vitreous cavity. In embodiments, the para-retinal administration comprises injecting at a distance of between 0-5 mm from the surface of the retina in the posterior vitreous cavity. In embodiments, the para-retinal administration comprises injecting at a distance of between 0-3 mm from the surface of the retina in the posterior vitreous cavity.

In embodiments, the subject is a human.

In one aspect, the disclosure provides nucleic acids encoding an AAV capsid protein comprising a VP3 portion, wherein the VP3 portion comprises variable regions (VR) I to IX wherein:

(a) VR-II comprises amino acid sequence
(SEQ ID NO: 54)
DNNGVK,
(b) VR-III comprises amino acid sequence
(SEQ ID NO: 55)
NDGS,
(c) VR-IV comprises amino acid sequence
(SEQ ID NO: 171)
QSTGGTAGTQQ,
(d) VR-V comprises amino acid sequence
(SEQ ID NO: 57)
RVSTTTGQNNNSNFAWTA,
(e) VR-VI comprises amino acid sequence
(SEQ ID NO: 58)
HKEGEDRFFPLSG,
(f) VR-VII comprises amino acid sequence
(SEQ ID NO: 59)
KQNAARDNADYSDV,
(g) VR-VIII comprises amino acid sequence
(SEQ ID NO: 60)
ADNLQQQNTAPQI,
and
(h) VR-IX comprises amino acid sequence
(SEQ ID NO: 61)
NYYKSTSVDF.

In embodiments, the VR-I region comprises NGTSGGST (SEQ ID NO: 170). In embodiments, the VP3 portion has the amino acid sequence of SEQ ID NO: 166. In embodiments, the AAV capsid protein further comprises i) a VP2 portion or ii) a VP1 portion and a VP2 portion.

In embodiments, the encoded AAV capsid protein comprises an amino acid sequence at least 95%, at least 96%, at least 97%, at least 98%, at least 99%, or 100% identical to SEQ ID NO: 164 or an amino acid sequence having up to 1, 2, 3, 4, 5, 6, 7, 8, 9, or 10 amino acids different from SEQ ID NO: 164. In embodiments, the encoded AAV capsid protein comprises an amino acid sequence at least 95%, at least 96%, at least 97%, at least 98%, at least 99%, or 100% identical to SEQ ID NO: 165 or an amino acid sequence having up to 1, 2, 3, 4, 5, 6, 7, 8, 9, or 10 amino acids different from SEQ ID NO: 165. In embodiments, the encoded AAV capsid protein comprises an amino acid sequence at least 95%, at least 96%, at least 97%, at least 98%, at least 99%, or 100% identical to SEQ ID NO: 166 or an amino acid sequence having up to 1, 2, 3, 4, 5, 6, 7, 8, 9, or 10 amino acids different from SEQ ID NO: 166. In embodiments, the nucleic acid sequence is at least 95% identical to the nucleotide sequence selected from SEQ ID NOs: 167-169. In embodiments, the nucleic acid sequence is 100% identical to the nucleotide sequence selected from SEQ ID NOs: 167-169.

In one aspect, the disclosure provides vectors comprising the nucleic acid of the disclosure.

In one aspect, the disclosure provides AAV capsid proteins encoded by the nucleic acid of the disclosure. In embodiments, the protein comprises the amino acid sequence of SEQ ID NO: 164, 165 or 166, or an amino acid sequence having up to 1, 2, 3, 4, 5, 6, 7, 8, 9, or 10 amino acids different from SEQ ID NO: 164, 165 or 166.

In one aspect, the disclosure provides AAV viral vectors comprising the AAV capsid protein encoded by the nucleic acid of the disclosure and an AAV vector genome, wherein the AAV vector genome comprises, in 5′ to 3′ orientation:
(a) a first AAV inverted terminal repeat,
(b) a promoter,
(c) a heterologous nucleic acid,
(d) a polyadenylation signal, and
(e) a second AAV inverted terminal repeat.

In embodiments, the heterologous nucleic acid is operably linked to a constitutive promoter. In embodiments, the heterologous nucleic acid encodes a polypeptide. In embodiments, the heterologous nucleic acid encodes an antisense RNA, an siRNA, a microRNA, or a gRNA. In embodiments, the AAV capsid protein comprises the amino acid sequence of SEQ ID NO: 164, 165 or 166.

In one aspect, the disclosure provides AAV viral vectors comprising
(i) an AAV capsid protein having the amino acid sequence of SEQ ID NO: 164 and
(ii) an AAV vector genome, wherein the AAV vector genome comprises, in 5′ to 3′ orientation:
   (a) a first AAV inverted terminal repeat,
   (b) a promoter,
   (c) a heterologous nucleic acid,
   (d) a polyadenylation signal; and
   (e) a second AAV inverted terminal repeat.

In embodiments, the heterologous nucleic acid encodes a polypeptide having at least 90% identity to any one of SEQ ID NOs: 142-144 and 177-181. In embodiments, the heterologous nucleic acid comprises a polynucleotide sequence having at least 90% identity to any one of SEQ ID NOs: 116-118 and 172-176.

In one aspect, the disclosure provides methods of treating a disease or disorder comprising administering the AAV viral vector of the disclosure to a subject. In embodiments, the AAV viral vector is administered to the subject orally, rectally, transmucosally, inhalationally, transdermally, parenterally, intravenously, subcutaneously, intradermally, intramuscularly, intrapleurally, intracerebrally, intrathecally, intracerebrally, intraventricularly, intranasally, intra-aurally, intra-ocularly, peri-ocularly, topically, intralymphatically, intracistemally, intravitreally, para-retinally, or sub-retinally. In embodiments, the disease or disorder is amyotrophic lateral sclerosis (ALS), spinal muscular atrophy (SMA), Fabry disease, Pompe disease, CLN3 disease (or Juvenile Neuronal Ceroid Lipofuscinosis), recessive dystrophic epidermolysis bullosa (RDEB), juvenile Batten disease, autosomal dominant disorder, muscular dystrophy, hemophilia A, hemophilia B, multiple sclerosis, diabetes mellitus, Gaucher disease cancer, arthritis, muscle wasting, heart disease, intimal hyperplasia, epilepsy, Huntington's disease, Parkinson's disease, Alzheimer's disease, cystic fibrosis, thalassemia, Hurler's Syndrome, Sly syndrome, Scheie Syndrome, Hurler-Scheie Syndrome, Hunter's Syndrome, Sanfilippo Syndrome A (mucopolysaccharidosis IIIA or MPS IIIA), Sanfilippo Syndrome B (mucopolysaccharidosis IIIB or MPS IIIB), Sanfilippo Syndrome C, Sanfilippo Syndrome D, Morquio Syndrome, Maroteaux-Lamy Syndrome, Krabbe's disease, phenylketonuria, Batten's disease, spinal cerebral ataxia, LDL receptor deficiency, hyperammonemia, arthritis, macular degeneration, retinitis pigmentosa, ceroid lipofuscinosis, neuronal, 1 (CLN1), adenosine deaminase deficiency, Dominant Optic Atrophy, Retinoschisis, Stargardt disease, Bietti's Crystalline Dystrophy or BEST vitelliform macular dystrophy. In embodiments, the diseases or disorder is an ophthalmic disease or disorder. In embodiments, the ophthalmic disease or disorder is selected from the group consisting of dominant optic atrophy, retinitis pigmentosa, macular degeneration, an eye disorder related to mutations in the bestrophin-1 (BEST-1) gene, Leber congenital amaurosis, cone-rod dystrophy, Stargardt disease, choroideremia, Usher Syndrome, retinoschisis, Bietti's Crystalline Dystrophy and Achromatopsia. In embodiments, the subject is a human.

### BRIEF DESCRIPTION OF THE DRAWINGS

FIG. 1 is an illustration of the various modes of intraocular administrations. Adapted from Yiu et al., Mol Ther Methods Clin Dev. 2020 Jan. 21; 16:179-191, the content of which is incorporated by reference herein in its entirety.

FIGS. 2A-2E show AAV viral vector-mediated GFP expression in the eyes of a non-human primate animal model via intravitreal or para-retinal administrations. Scanning laser ophthalmoscopy (SLO) imaging was performed at Day 26 after injection of the indicated AAV viral vector. FIG. 2A shows transduction spread mediated by intravitreal injection of AAV viral vector comprising AAV204 capsid protein. FIG. 2B shows transduction spread mediated by para-retinal injection of AAV viral vector comprising AAV204 capsid protein. FIG. 2C shows transduction spread mediated by para-retinal injection of AAV viral vector comprising AAV8 capsid protein. FIG. 2D shows transduction spread mediated by para-retinal injection of AAV viral vector comprising AAV214 capsid protein. FIG. 2E shows transduction spread mediated by para-retinal injection of AAV viral vector comprising AAV214-D5 capsid protein.

FIGS. 3A-3F show imaging analysis of retinas after AAV administration. FIG. 3A shows the composite images of retina after AAV204 intravitreal administration. FIG. 3B shows the composite (upper left), rhodopsin (upper right), and zoom-in composite (lower) images of retina after AAV204 para-retinal administration. FIG. 3C shows the composite (upper left), rhodopsin (upper right), and zoom-in composite (lower) images of retina after AAV204 para-retinal administration. FIG. 3D shows the immunohistochemistry analysis of rhodopsin and GFP expression 1-month post para-retinal injection of AAV204 or AAV8 viral vector. FIG. 3E shows rhodopsin and GFP expression in the fovea post para-retinal injection of AAV204 viral vector. FIG. 3F shows rhodopsin and GFP expression along the papillomacular bundle post para-retinal injection of AAV204 viral vector.

FIGS. 4A-4C show AAV viral vector-mediated GFP expression in the eyes of a non-human primate animal model via sub-retinal administration. SLO imaging was performed at Day 27 after injection of the indicated AAV viral vector. FIG. 4A shows transduction spread mediated by sub-retinal injection of AAV viral vector comprising AAV8 capsid protein. FIG. 4B shows transduction spread mediated by sub-retinal injection of AAV viral vector comprising AAV214 capsid protein. FIG. 4C shows transduction spread mediated by sub-retinal injection of AAV viral vector comprising AAV214-D5 capsid protein. FIG. 4D shows the composite (upper left), rhodopsin (upper right), and zoom-in composite (lower) images of retina after AAV8 subretinal administration. FIG. 4E shows the composite (upper left), rhodopsin (upper right), and zoom-in composite (lower) images of retina after AAV214 subretinal administration. FIG. 4F shows the composite (upper left), rhodopsin (upper right), and zoom-in composite (lower) images of retina after AAV214-D5 subretinal administration.

FIG. 5 shows a diagram of the VP1, VP2, and VP3 portions of the capsid protein. The VP1- and VP2-specific portions are indicated along with the VP3 portion, which is identical to the VP3 protein produced. The amino acid sequence of AAV214 VP3 (SEQ ID NO: 41) is shown and variable regions I-IX are indicated. The full VP1 protein amino acid sequence for AAV214 is provided as SEQ ID NO: 3.

FIG. 6 shows an alignment of the VP1 protein amino acid sequences of AAV214 (SEQ ID NO: 3) and AAV214-D5 (SEQ ID NO: 164).

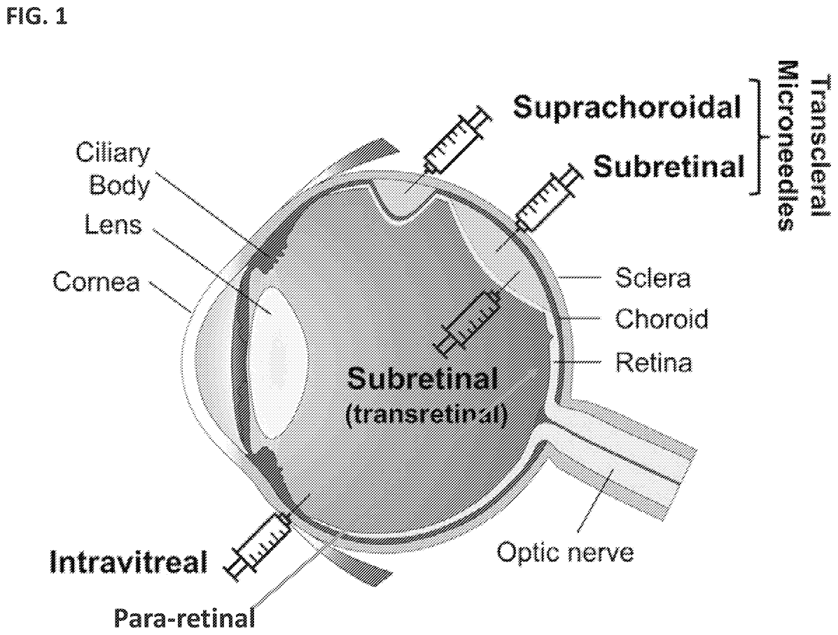

### DETAILED DESCRIPTION

Some embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

Unless the context indicates otherwise, it is specifically intended that the various features of the invention described herein can be used in any combination. Moreover, the disclosure also contemplates that in embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols in Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (RI. Freshney, ed. (1987)).

It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 10% of the specified amount.

The terms “acceptable”, “effective”, or “sufficient” when used to describe the selection of any components, ranges, dose forms, etc. disclosed herein intend that said component, range, dose form, etc. is suitable for the disclosed purpose.

Unless specifically recited, the term “host cell” includes a eukaryotic host cell, including, for example, fungal cells, yeast cells, higher plant cells, insect cells and mammalian cells. Non-limiting examples of eukaryotic host cells include simian, bovine, porcine, murine, rat, avian, reptilian and human, e.g., HEK293 cells and 293T cells.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

“Under transcriptional control” is a term well understood in the art and indicates that transcription of a polynucleotide sequence, usually a DNA sequence, depends on its being operatively linked to an element that contributes to the initiation of, or promotes, transcription. “Operatively linked” intends that the polynucleotides are arranged in a manner that allows them to function in a cell. In one aspect, this invention provides promoters operatively linked to the downstream sequences.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide and/or a fragment thereof. The antisense strand is the complement of such a nucleic acid, and the encoding sequence can be deduced therefrom.

The term “promoter” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, an H1 promoter, a ubiquitous chicken β-actin hybrid (CBh) promoter, a small nuclear RNA (U1a or U1b) promoter, an MeCP2 promoter, an MeP418 promoter, an MeP426 promoter, a minimal MeCP2 promoter, a VMD2 promoter, an mRho promoter or an EFI promoter.

Additional non-limiting exemplary promoters provided herein include, but are not limited to EF1a, Ubc, human β-actin, CAG, TRE, Ac5, Polyhedrin, CaMKIIa, Gal1, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In embodiments, the promoter is used together with an enhancer to increase the transcription efficiency. Non-limiting examples of enhancers include an interstitial retinoid-binding protein (IRBP) enhancer, an RSV enhancer or a CMV enhancer.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise, consist essentially of, or consist of a protein's or peptide's sequence. As used herein the term “amino acid” refers to either natural and/or unnatural or synthetic amino acids, including glycine and both the D and L optical isomers, amino acid analogs and peptidomimetics.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In embodiments, the signal peptide is removed following localization. Examples of signal peptides are well known in the art. Non-limiting examples are those described in U.S. Pat. Nos. 8,853,381, 5,958,736, and 8,795,965. In embodiments, the signal peptide can be an IDUA signal peptide.

The terms “equivalent” or “biological equivalent” are used interchangeably when referring to a particular molecule, biological material, or cellular material and intend those having minimal homology while still maintaining desired structure or functionality. Non-limiting examples of equivalent polypeptides include a polypeptide having at least about 60%, at least about 65%, at least about 70%, at least about 75%, at least about 80%, at least about 85%, at least about 90%, at least about 95% identity or at least about 99% identity to a reference polypeptide (for instance, a wild-type polypeptide); or a polypeptide which is encoded by a polynucleotide having at least about 70%, at least about 75%, at least about 80%, at least about 85%, at least about 90%, at least about 95% identity, at least about 97% sequence identity or at least about 99% sequence identity to the reference polynucleotide (for instance, a wild-type polynucleotide).

“Homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using ClustalW (available at https://genome.jp/tools-bin/clustalw/) and Gonnet (for protein) weight matrix. In embodiments, the ClustalW parameters used for performing nucleic acid sequence alignments using the nucleic acid sequences found herein are generated using the ClustalW (for DNA) weight matrix.

As used herein, amino acid modifications may be substitutions, deletions or insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g., charge, hydrophobicity or size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one charged or polar residue for another, such as the substitution of arginine for lysine, glutamic acid for aspartic acid, glutamine for asparagine, and the like. Other illustrative examples of conservative substitutions include the changes of: alanine to serine; asparagine to glutamine or histidine; aspartate to glutamate; cysteine to serine; glycine to proline; histidine to asparagine or glutamine; lysine to arginine, glutamine, or glutamate; phenylalanine to tyrosine, serine to threonine; threonine to serine; tryptophan to tyrosine; tyrosine to tryptophan or phenylalanine; and the like.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally-occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

A “viral vector” is defined as a recombinantly produced virus or viral particle that contains a polynucleotide to be delivered into a host cell, either in vivo, ex vivo or in vitro. Examples of viral vectors include retroviral vectors, AAV viral vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5(7):823-827.

As used herein, the term “recombinant expression system” or “recombinant vector” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; bacteria; viruses, such as baculoviruses, adenoviruses and retroviruses; bacteriophage, cosmid, plasmid, and fungal vectors; and other recombination vehicles typically used in the art which have been described for expression in a variety of eukaryotic and prokaryotic hosts, and may be used for gene therapy as well as for simple protein expression. Liposomes that also comprise, consist essentially of, or consist of a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized prior to introduction into the host cell.

“Plasmids” used in genetic engineering are called “plasmid vectors”. Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, consisting essentially of, or consisting of the viral genome or part thereof, and a transgene.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In embodiments, the AAV refers to the serotype AAV1, AAV2, AAV4, AAV5, AAV6, AAV7, AAV8, AAV9, AAV10, AAV11, AAV12, AAV13, AAVPHP.B, or AAVrh74.

An “AAV vector” as used herein refers to a vector comprising one or more heterologous nucleic acid (HNA) sequences and one or more AAV inverted terminal repeat sequences (ITRs). Such AAV vectors can be replicated when present in a host cell that provides the functionality of rep and cap gene products, and allow the ITRs and the nucleic acid between the ITRs to be packaged into infectious viral particles. In embodiments, AAV vectors comprise a promoter, at least one nucleic acid that may encode at least one protein or RNA, and/or an enhancer and/or a terminator within the flanking ITRs that is packaged into the infectious AAV particle. The ITRs and the nucleic acid between the ITRs can be encapsidated into the AAV capsid, and this encapsidated portion of the nucleic acid may be referred to as the “AAV vector genome.” AAV vectors may contain elements in addition to the encapsidated portion, for example, antibiotic resistance genes or other elements known in the art included in the plasmid for manufacturing purposes but not packaged into the AAV particle.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

An “AAV virion” or “AAV viral particle” or “AAV viral vector” or “AAV vector particle” or “AAV particle” refers to a viral particle composed of at least one AAV capsid protein and an encapsidated AAV vector genome.

As used herein, the term “helper” in reference to a virus or plasmid refers to a virus or plasmid used to provide the additional components necessary for replication and packaging of any one of the AAV vector genomes disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In embodiments, the pHELP plasmid may be the pHELPK plasmid, wherein the ampicillin expression cassette is exchanged with a kanamycin expression cassette; pHELPK has the sequence shown in SEQ ID NO: 92.

As used herein, a packaging cell (or a helper cell) is a cell used to produce viral vectors. Producing recombinant AAV viral vectors requires Rep and Cap proteins provided in trans as well as gene sequences from Adenovirus that help AAV replicate. In some aspects, Packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

As used herein, a reporter protein is a detectable protein that is operably linked to a promoter to assay the expression (for example, tissue specificity and/or strength) of the promoter. In aspects, a reporter protein may be operably linked to a polypeptide. In aspects, reporter proteins may be used in monitoring DNA delivery methods, functional identification and characterization of promoter and enhancer elements, translation and transcription regulation, mRNA processing and protein: protein interactions. Non-limiting examples of a reporter protein are β-galactosidase; a fluorescent protein, such as, Green Fluorescent Protein (GFP) or Red Fluorescent Protein (RFP); luciferase; glutathione S-transferase; and maltose binding protein.

A “pharmaceutical composition” is intended to include the combination of an active ingredient—such as a polypeptide, a polynucleotide, an antibody or a viral vector—and a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject” of diagnosis or treatment is a cell or an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In embodiments, the subject is a human.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected), or a group of tissues making up an organ or part or region of the body of an organism. The tissue may comprise, consist essentially of, or consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, in embodiments the effective amount is the amount sufficient to result in regaining part or full function of a gene that is deficient in a subject. In embodiments, the effective amount of an AAV viral particle is the amount sufficient to result in expression of a gene in a subject. The skilled artisan will be able to determine appropriate amounts depending on these and other factors.

In embodiments, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

### AAV Structure and Function

AAV is a replication-deficient parvovirus, the single-stranded DNA genome of which is about 4.7 kb in length, including two about 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively, the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference in its entirety. U.S. Pat. No. 9,434,928 also provide the sequences of the capsid proteins and a self-complementary genome. In one aspect, the genome is a self-complementary genome. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threoninc) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus polyadenylation signal is located at map position 95 of the AAV genome. The life cycle and genetics of AAV are reviewed in Muzyczka, Current Topics in Microbiology and Immunology, 158: 97-129 (1992).

Each VP1 protein contains a VP1 portion, a VP2 portion and a VP3 portion. The VP1 portion is the N-terminal portion of the VP1 protein that is unique to the VP1 protein, corresponding to the amino acids 1-137 portion of SEQ ID NO: 164. The VP2 portion is the amino acid sequence present within the VP1 protein that is also found in the N-terminal portion of the VP2 protein, corresponding to the amino acids 138-202 portion of SEQ ID NO: 164. The VP3 portion and the VP3 protein have the same sequence. The VP3 portion is the C-terminal portion of the VP1 protein that is shared with the VP1 and VP2 proteins, corresponding to the amino acids 203-737 portion of SEQ ID NO: 164. See FIG. 5.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are incorporated herein by reference.

AAV possesses unique features that make it attractive as a viral vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vector genomes. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise, consist essentially of, or consist of a nucleic acid molecule encoding a therapeutic protein (e.g., CYP4V2, RS1, PDE6B, ABCA4, BEST1, OPA1 or OPA3) and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV PHP.B and AAV rh74. Production of pseudotyped rAA Vis disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

### AAV Vector Particles, Capsid Proteins, and AAV Vectors

Provided herein are AAV vector particles, AAV vectors, and capsid proteins that have desirable tissue specificity and find use in delivering a variety of therapeutic payloads, including nucleic acids, and proteins useful in the treatment of disease.

### AAV Capsid Proteins

The disclosure provides AAV particles possessing properties of high gene transfer efficiency and increased tissue tropism. AAV viral vector delivery currently relies on the use of serotype selection for tissue targeting based on the natural tropism of the virus or by the direct injection into target tissues. Many currently available AAV viral vectors are, however, suboptimal for delivering genes to a specific target site.

The present disclosure provides AAV capsid protein sequences that confer high gene transfer efficiency and increased tissue specificity on the AAV particles comprising them. In embodiments, the AAV particles comprising such AAV capsid proteins are administered via a specific delivery route to achieve optimal delivery to specific target site.

In embodiments, the VP1 capsid protein comprises any one of the amino acid sequences listed in Table 1, or a sequence having up to 1, 2, 3, 4, 5, 6, 7, 8, 9, or 10 amino acids mutated, deleted or added as compared to, any one of the amino acid sequences listed in Table 1. In embodiments, up to 15 amino acids, up to 20 amino acids, up to 30 amino acids, or up to 40 amino acids may be mutated, deleted or added compared to these sequences, In embodiments, the VP1 capsid protein is encoded by any one of the nucleic acid sequences listed in Table 1, or a sequence having up to 5, up to 10, up to 30, or up to 60 nucleotide changes to any one of the nucleic acid sequences listed in Table 1.

TABLE 1
VP1 Capsid Proteins

| Amino Acid SEQ ID NO: | NA SEQ ID NO: | AAV Capsid Name |
|---|---|---|
| 1 | 98 | AAV 110 |
| 2 | 15 | AAV 204 |
| 3 | 18 | AAV 214 |
| 30 | 19 | AAV 214A |
| 31 | 20 | AAV 214e |
| 32 | 21 | AAV 214e8 |
| 33 | 22 | AAV 214e9 |
| 34 | 23 | AAV 214e10 |
| 49 | 47 | AAV ITB102_45 |
| 84 | 82 | AAV 214AB |
| 164 | 167 | AAV 214-D5 |

In embodiments, the AAV VP1 protein comprises, consists essentially of, or consists of an amino acid sequence of SEQ ID NOs: 1-3, 30-34, 49, 84 or 164, or a sequence having up to 1, 2, 3, 4, 5, 6, 7, 8, 9, or 10 amino acids different from SEQ ID NOs: 1-3, 30-34, 49, 84 or 164. Also provided are polynucleotides encoding these VP1 proteins. In embodiments, the polynucleotides encoding the VP1 proteins comprise, consist essentially of, or consist of the sequence of SEQ ID NOs: 15, 18-23, 47, 82, 98 or 167 or a sequence having up to 5, up to 10, or up to 30 nucleotide changes to SEQ ID NOs: 15, 18-23, 47, 82, 98 or 167.

In embodiments, the AAV capsid sequence is an AAV-110 capsid protein (SEQ ID NO: 1), AAV204 capsid protein (SEQ ID NO: 2), AAV214 capsid protein (SEQ ID NO: 3) or AAV ITB102_45 capsid protein (SEQ ID NO: 49). In embodiments, the AAV capsid protein is a variant of the AAV214 capsid protein. In embodiments, the AAV capsid protein is AAV214A (SEQ ID NO: 30), AAV-214-AB (SEQ ID NO: 84), AAV214e (SEQ ID NO: 31), AAV214e8 (SEQ ID NO: 32), AAV214e9 (SEQ ID NO: 33), AAV214e10 (SEQ ID NO: 34), or AAV214-D5 (SEQ ID NO: 164). In embodiments, the AAV capsid protein is AAV214-D5 (SEQ ID NO: 164).

In embodiments, the AAV capsid sequence is an AAV204 capsid protein (SEQ ID NO: 2), AAV214 capsid protein (SEQ ID NO: 3), AAV214-D5 capsid protein (SEQ ID NO: 164) or AAV8 capsid protein (SEQ ID NO: 67).

Sequences for exemplary VP2 and VP3 proteins are provided in Table 2 and Table 3. Given the VP2 and VP3 sequences, the VP1 portions may be determined by alignment with the full, VP1 protein sequence.

TABLE 2
VP2 Capsid Proteins

| Amino Acid SEQ ID NO: | Name |
|---|---|
| 35 | 214 |
| 36 | 214A |
| 37 | 214e |
| 38 | 214e8 |
| 39 | 214e9 |
| 40 | 214e10 |
| 85 | 214AB |
| 50 | ITB102_45 |
| 165 | 214-D5 |

In embodiments, the AAV VP2 proteins comprise, consist essentially of, or consist of an amino acid sequence of any one of SEQ ID NOs: 35-40, 50, 85 and 165, or a sequence having up to 1, 2, 3, 4, 5, 6, 7, 8, 9, or 10 amino acids different from SEQ ID NOs: 35-40, 50, 85 or 165. In embodiments, the AAV VP2 proteins comprise, consist essentially of, or consist of an amino acid sequence of SEQ ID NO: 165, or a sequence having up to 1, 2, 3, 4, 5, 6, 7, 8, 9, or 10 amino acids different from SEQ ID NO: 165.

Also provided are polynucleotides encoding these VP2 proteins. In embodiments, the polynucleotide encoding the VP2 protein comprises, consists essentially of, or consists of the sequence of SEQ ID NO: 47, or a sequence having up to 5, up to 10, or up to 30 nucleotide changes to SEQ ID NO: 47. In embodiments, the polynucleotide encoding the VP2 protein comprises, consists essentially of, or consists of the sequence of SEQ ID NO: 168, or a sequence having up to 5, up to 10, or up to 30 nucleotide changes to SEQ ID NO: 168.

Exemplary nucleic acids for the other capsid VP2 portions may be derived from the corresponding portions of the VP1 capsid protein nucleic acids.

TABLE 3
VP3 Capsid proteins

| Amino Acid SEQ ID NO: | NA SEQ ID NO: | AAV Capsid Name |
|---|---|---|
| 17 | 16 | 204 |
| 41 | 24 | 214 |
| 42 | 25 | 214A |
| 43 | 26 | 214e |
| 44 | 27 | 214e8 |
| 45 | 28 | 214e9 |
| 46 | 29 | 214e10 |
| 86 | 83 | 214AB |
| 51 | 48 | ITB102_45 |
| 166 | 169 | 214-D5 |

The VP3 proteins of AAV214, AAV214e, AAV214e8, AAV214e9, AAV214e10 have the same amino acid (SEQ ID NO:41) and nucleic acid (SEQ ID NO: 24) sequences.

In embodiments, the AAV VP3 proteins comprise, consist essentially of, or consist of the amino acid sequence of SEQ ID NOs: 17, 41-46, 51, 86, or 166, or a sequence having up to 1, 2, 3, 4, 5, 6, 7, 8, 9, or 10 amino acids different from SEQ ID NOs: 17, 41-46, 51, 86, or 166. In embodiments, the AAV VP3 proteins comprise, consist essentially of, or consist of the amino acid sequence of SEQ ID NO: 166, or a sequence having up to 1, 2, 3, 4, 5, 6, 7, 8, 9, or 10 amino acids different from SEQ ID NO: 166.

Also provided are polynucleotides encoding these VP3 proteins. In embodiments, the polynucleotides encoding the proteins that comprise, consist essentially of, or consist of the sequence of SEQ ID NOs: 16, 24-29, 48, 83 or 169, or a sequence having up to 5, up to 10, or up to 30 nucleotide changes to SEQ ID NOs: 16, 24-29, 48, 83, or 169. In embodiments, the polynucleotides encoding the proteins that comprise, consist essentially of, or consist of the sequence of SEQ ID NO: 169, or a sequence having up to 5, up to 10, or up to 30 nucleotide changes to SEQ ID NO: 169.

In embodiments, the AAV capsid protein is a chimeric protein. In embodiments, a VP1, VP2, or VP3 portion of the AAV capsid protein disclosed herein may be replaced with a VP1, VP2, or VP3 portion from a different AAV capsid protein disclosed herein.

In embodiments, provided herein is an AAV capsid protein comprising a leucine residue at amino acid 129, an asparagine residue at amino acid 586 and a glutamic acid residue at amino acid 723, wherein amino acid positions in the AAV capsid protein are numbered with respect to amino acid positions in the amino acid sequence of SEQ ID NO: 2. In some cases, the protein comprises the amino acid sequence of SEQ ID NO: 2. In other cases, these amino acids may be introduced into other capsid proteins.

In embodiments, provided herein is an AAV VP1 capsid protein comprising a VP1 portion, a VP2 portion and a VP3 portion, wherein the VP1 portion comprises a leucine (L) residue at amino acid 129, wherein the VP2 portion comprises a threonine (T) or asparagine (N) residue at amino acid 157 and a lysine (K) or serine (S) residue at amino acid 162, and wherein the VP3 portion comprises asparagine (N) residue at amino acid 223, an alanine (A) residue at amino acid 224, a histidine (H) residue at amino acid 272, a threonine (T) residue at amino acid 410, a histidine (H) residue at amino acid 724 and a proline (P) residue at amino acid 734, wherein amino acid positions in the AAV capsid protein are numbered with respect to amino acid positions in the amino acid sequence of SEQ ID NO: 3 (i.e., VP1 capsid subunit numbering).

In embodiments, the VP1 portion further comprises an aspartic acid (D) or alanine (A) residue at amino acid 24, wherein amino acid positions in the AAV capsid protein are numbered with respect to amino acid positions in the amino acid sequence of SEQ ID NO: 3. In embodiments, the VP2 portion further comprises one or more of (i) a proline (P) residue at amino acid 148; (ii) an arginine (R) residue inserted at amino acid 152; (iii) an arginine (R) residue at amino acid 168; (iv) an isoleucine (I) residue at amino acid 189; and (v) a serine (S) residue at amino acid 200, wherein amino acid positions in the AAV capsid protein are numbered with respect to amino acid positions in the amino acid sequence of SEQ ID NO: 3.

In embodiments, one or more variable regions I through IX (see FIG. 5) in the disclosed VP3 portion capsid proteins may be removed and replaced with alternative regions. Suitable alternatives are identified in Table 6 below. The location for these, as well as the identity of additional alternatives may be identified by alignment to SEQ ID NO:41 as shown in FIG. 5. In embodiments, one or more VRs may have an insertion of 1, 2 or 3 amino acids. In embodiments, one or more VRs may have a deletion of 1, 2 or 3 amino acids.

TABLE 6
Variable Regions

| VR | Sequence |
|---|---|
| I | SASTGAS (SEQ ID NO. 52); NSTSGGSS (SEQ ID NO. 53); |
| | SSTSGGSS (SEQ ID NO: 87); NGTSGGST (SEQ ID NO: 170) |
| II | DNNGVK (SEQ ID NO. 54) |
| III | NDGS (SEQ ID NO. 55) |
| IV | INGSGQNQQT (SEQ ID NO. 56); QSTGGTAGTQQ (SEQ ID NO: |
| | 171) |
| V | RVSTTTGQNNNSNFAWTA (SEQ ID NO. 57) |
| VI | HKEGEDRFFPLSG (SEQ ID NO. 58) |
| VII | KQNAARDNADYSDV (SEQ ID NO: 59) |
| VIII | ADNLQQQNTAPQI (SEQ ID NO. 60) |
| IX | NYYKSTSVDF (SEQ ID NO. 61). |

The disclosure provides nucleic acids encoding any one of the AAV capsid proteins disclosed herein. The disclosure also provides vectors comprising any one of the nucleic acids disclosed herein.

In embodiments, AAV is an AAV9 serotype. Alternative serotypes or modified capsid viruses can be used to optimize neuronal tropism. Alternative vectors include: a modified AAV9 serotype vector for higher neuronal tropism than standard AAV9, e.g., PHP.B that uses a Cre-lox recombination system to identify neuronally targeted vectors. Alternatively, the AAV9 PHP.B has a modified amino acid 498 of VP1 from asparagine to lysine to reduce the liver tropism. Further variants of AAVrh74 that have mutated several amino acids can be used for very broad tissue tropism including the brain.

### AAV Vectors

The AAV vectors supply the nucleic acid that becomes encapsidated into the AAV vector particle including element(s) involved in controlling expression of the nucleic acids in the subject, as well as the ITRs to facilitate encapsidation. In embodiments, the AAV vectors disclosed herein comprise at least one heterologous nucleic acid (HNA) sequence, which, when expressed in a cell of a subject, is effective to treat a disease or disorder. In embodiments, the HNA sequence comprises a transgene. In embodiments, the AAV vectors comprise at least one ITR sequence and at least one transgene. In embodiments, the transgene encodes a therapeutic protein or a therapeutic RNA.

In embodiments, control of transgene expression in the host cell may be regulated by regulatory elements contained within the AAV vector, including promoter sequences, and polyadenylation signals. In embodiments, the AAV vector may also encode a signal peptide. In embodiments, the AAV vectors have 5′ and 3′ inverted terminal repeats (ITRs). The 5′ ITR is located upstream of a promoter, which in turn is upstream of the transgene. In embodiments, the 5′ and 3′ ITR have the same sequence. In embodiments, they have a different sequence. In embodiments, an AAV vector of the disclosure may comprise, in 5′ to 3′ orientation, a first (5′) ITR, a promoter, a transgene, a polyadenylation signal, and a second (3′) ITR.

In embodiments, the HNA (for example, an HNA comprising a transgene) is operably linked to a constitutive promoter. The constitutive promoter can be any constitutive promoter known in the art and/or provided herein. In embodiments, the constitutive promoter comprises, consists essentially of, or consists of a Rous sarcoma virus (RSV) LTR promoter (optionally with the RSV enhancer), a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a hybrid chicken beta actin promoter, a MeCP2 promotor, an H1 promoter, a U1a promoter, a mMeP418 promoter, a mMeP426 promoter, a minimal MeCP2 promoter, a CAG promoter, or an EF1 promoter. It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). In embodiments, the HNA sequence is operably linked to a tissue-specific control promoter, or an inducible promoter. In embodiments, the tissue-specific control promoter is a central nervous system (CNS) cell-specific promoter, a lung-specific promoter, a skin-specific promoter, a muscle-specific promoter, a liver-specific promoter, an eye-specific promoter (e.g., a VMD2, or mRho promoter).

In embodiments, the promoter may comprise, consist essentially of or consist of a polynucleotide having the sequence of SEQ ID NO: 96 (mouse U1 promoter) or a SEQ ID NO: 97 (a H1 promoter). In embodiments, the promoter is an U1a or U1b promoter, EF1 promoter, or CBA (chicken beta-actin). In embodiments, the promoter may comprise, consist essentially of or consist of any one of the nucleic acid sequences listed in Table 5, or a sequence having up to 5, up to 10, up to 20, or up to 30 nucleotide changes to any one of the nucleic acid sequences listed in Table 5. In embodiments, the promoter may comprise, consist essentially of, or consist of a nucleic acid sequence having at least 90%, at least 95%, at least 97%, at least 98%, at least 99%, or 100% identity to any one of the nucleic acid sequences listed in Table 5.

TABLE 5
Non-limiting Examples of Promoters

| | Nucleic acid |
|---|---|
| Promoter name | SEQ ID No. |
| Mouse U1a promoter | 152 |
| Polymerase III H1 mutant promoter | 153 |
| Chicken β-actin hybrid promoter CBh | 154 |
| (CBh promoter consists | |
| of CMV enhancer, CBA promoter, | |
| first CBA exon and partial intron) | |
| MeCP2 min promoter sequence | 155 |
| MeCP2 promoter sequence | 156 |
| MeCP418 promoter sequence | 157 |
| MeCP426 promoter sequence | 158 |
| VMD2 promoter | 159 |
| PDE6b promoter | 160 |
| mRho promoter | 161 |
| CMV promoter | 162 |
| UbC promoter | 163 |

In embodiments, the HNA sequence is operably liked to an additional regulatory element. The additional regulatory element can be a woodchuck hepatitis virus post-transcriptional regulatory element (“WPRE”). In embodiments, the AAV vector may comprise regulatory components suitable for growth and culture of the vector in a bacterial host for vector production purposes. For example, the vector may comprise genes for antibiotic resistance, and maintenance of the plasmid in bacteria, as well as associated regulatory elements to control protein expression in bacteria.

In embodiments, the HNA sequence is operably linked to a polyadenylation signal. In embodiments, the polyadenylation signal comprises, consists essentially of or consists of an MeCP2 polyadenylation signal, a retinol dehydrogenase 1 (RDH1) polyadenylation signal, a bovine growth hormone (BGH) polyadenylation signal, an SV40 polyadenylation signal, a SPA49 polyadenylation signal, a sNRP-TK65 polyadenylation signal, a sNRP polyadenylation signal, or a TK65 polyadenylation signal. An exemplary SPA49 polyadenylation signal is described in Ostedgaard et al., Proc. Nat'l Acad. Sci. USA (Feb. 22, 2005) 102:2952-2957, incorporated herein by reference.

### Heterologous Nucleic Acids (HNA)

The AAV viral vectors disclosed herein infect and deliver one or more heterologous nucleic acids (HNA) to target tissues. In embodiments, the HNA sequences are transcribed and optionally, translated in the cells of the target tissue.

In some cases, the HNA encodes an antisense RNA, microRNA, siRNA, or guide RNA (gRNA). CRISPR technology has been used to target the genome of living cells for modification Cas9 protein is a large enzyme that must be delivered efficiently to target tissues and cells to mediate gene repair through the CRISPR system and current CRISPR/Cas9 gene correction protocols suffer from a number of drawbacks. Long-term expression of Cas9 can elicit host immune responses. An additional guide RNA may be delivered via a separate vector due to packaging constraints. In embodiments, the HNA encodes a Cas9 protein or an equivalent thereof.

In embodiments, the HNA comprises a transgene encoding a protein, which may be expressed in cells of a subject to treat a disease or a disorder, resulting from reduced or eliminated activity of the native protein. Thus, in embodiments, the transgene may encode a protein selected from cystic fibrosis transmembrane conductance regulator (CFTR), N-acetyl-alpha-glucosaminidase (NAGLU), N-sulfoglucosamine sulfohydrolase (SGSH), palmitoyl-protein thioesterase 1 (PPT1), survival of motor neuron 1, telomeric (SMN1), alkaline phosphatase, biomineralization associated (ALPL, also known as TNALP), glial cell derived neurotrophic factor (GDNF), glucosylceramidase beta (GBA1), iduronidase alpha-L-(IDUA), methyl-CpG binding protein 2 (MeCP2), ceroid lipofuscinosis, neuronal, 1 (CLN1), rhodopsin (Rho), cytochrome P450 family 4 subfamily V member 2 (CYP4V2), retinoschisin 1 (RS1), phosphodiesterase 6B (PDE6B), ATP binding cassette subfamily A member 4 (ABCA4), Bestrophin-1 (BEST1), OPA1 Mitochondrial Dynamin Like GTPase (OPA1), and Optic Atrophy 3 (OPA3).

In embodiments, the transgene encodes a Cytochrome P450 family 4 subfamily V member 2 (CYP4V2). In embodiments, the CYP4V2 transgene comprises a mutant sequence, a codon-optimized sequence, and/or a truncated sequence of CYP4V2. In embodiments, the CYP4V2 transgene comprises, consists essentially of, or consists of a nucleic acid having the sequence of SEQ ID NO: 116, or a sequence having at least 80%, at least 85%, at least 90%, at least 95%, at least 97%, at least 98%, at least 99%, or at least 99.5% identity to SEQ ID NO: 116. In embodiments, the CYP4V2 transgene encodes a protein that comprises, consists essentially of, or consists of the amino acid sequence of SEQ ID NO: 142, or a sequence having at least 80%, at least 85%, at least 90%, at least 95%, at least 97%, at least 98%, at least 99%, or at least 99.5% identity to SEQ ID NO: 142. In embodiments, the AAV vector or AAV vector genome of the disclosure encodes CYP4V2 and is for treating Bietti's Crystalline Dystrophy.

In embodiments, the transgene encodes a Retinoschisin 1 (RS1). In embodiments, the RS1 transgene comprises a mutant sequence, a codon-optimized sequence, and/or a truncated sequence of RS1. In embodiments, the RS1 transgene comprises, consists essentially of, or consists of a nucleic acid having the sequence of SEQ ID NO: 117, or a sequence having at least 80%, at least 85%, at least 90%, at least 95%, at least 97%, at least 98%, at least 99%, or at least 99.5% identity to SEQ ID NO: 117. In embodiments, the RS1 transgene encodes a protein that comprises, consists essentially of, or consists of the amino acid sequence of SEQ ID NO: 143, or a sequence having at least 80%, at least 85%, at least 90%, at least 95%, at least 97%, at least 98%, at least 99%, or at least 99.5% identity to SEQ ID NO: 143. In embodiments, the AAV vector or AAV vector genome of the disclosure encodes RS1 and is for treating Retinoschisis.

In embodiments, the transgene encodes a Phosphodiesterase 6B (PDE6B). In embodiments, the PDE6B transgene comprises a mutant sequence, a codon-optimized sequence, and/or a truncated sequence of PDE6B. In embodiments, the PDE6B transgene comprises, consists essentially of, or consists of a nucleic acid having the sequence of SEQ ID NO: 118, or a sequence having at least 80%, at least 85%, at least 90%, at least 95%, at least 97%, at least 98%, at least 99%, or at least 99.5% identity to SEQ ID NO: 118. In embodiments, the PDE6B transgene encodes a protein that comprises, consists essentially of, or consists of the amino acid sequence of SEQ ID NO: 144, or a sequence having at least 80%, at least 85%, at least 90%, at least 95%, at least 97%, at least 98%, at least 99%, or at least 99.5% identity to SEQ ID NO: 144. In embodiments, the AAV vector or AAV vector genome of the disclosure encodes PDE6B and is for treating Retinitis pigmentosa.

In embodiments, the transgene encodes an ATP binding cassette subfamily A member 4 (ABCA4). In embodiments, the ABCA4 transgene comprises a mutant sequence, a codon-optimized sequence, and/or a truncated sequence of ABCA4. In embodiments, the ABCA4 transgene comprises, consists essentially of, or consists of a nucleic acid having the sequence of SEQ ID NO: 172, or a sequence having at least 80%, at least 85%, at least 90%, at least 95%, at least 97%, at least 98%, at least 99%, or at least 99.5% identity to SEQ ID NO: 172. In embodiments, the ABCA4 transgene encodes a protein that comprises, consists essentially of, or consists of the amino acid sequence of SEQ ID NO: 177, or a sequence having at least 80%, at least 85%, at least 90%, at least 95%, at least 97%, at least 98%, at least 99%, or at least 99.5% identity to SEQ ID NO: 177. In embodiments, the AAV vector or AAV vector genome of the disclosure encodes ABCA4 and is for treating Stargardt disease.

In embodiments, the transgene encodes a Bestrophin-1 (BEST-1). In embodiments, the BEST-1 transgene comprises a mutant sequence, a codon-optimized sequence, and/or a truncated sequence of BEST-1. In embodiments, the BEST-1 transgene comprises, consists essentially of, or consists of a nucleic acid having the sequence of SEQ ID NO: 173 or 174, or a sequence having at least 80%, at least 85%, at least 90%, at least 95%, at least 97%, at least 98%, at least 99%, or at least 99.5% identity to SEQ ID NO: 173 or 174. In embodiments, the BEST-1 transgene encodes a protein that comprises, consists essentially of, or consists of the amino acid sequence of SEQ ID NO: 178 or 179, or a sequence having at least 80%, at least 85%, at least 90%, at least 95%, at least 97%, at least 98%, at least 99%, or at least 99.5% identity to SEQ ID NO: 178 or 179. In embodiments, the AAV vector or AAV vector genome of the disclosure encodes BEST-1 and is for treating BEST vitelliform macular dystrophy.

In embodiments, the transgene encodes an OPA1 Mitochondrial Dynamin Like GTPase (OPA1). In embodiments, the OPA1 transgene comprises a mutant sequence, a codon-optimized sequence, and/or a truncated sequence of OPA1. In embodiments, the OPA1 transgene comprises, consists essentially of, or consists of a nucleic acid having the sequence of SEQ ID NO: 175, or a sequence having at least 80%, at least 85%, at least 90%, at least 95%, at least 97%, at least 98%, at least 99%, or at least 99.5% identity to SEQ ID NO: 175. In embodiments, the OPA1 transgene encodes a protein that comprises, consists essentially of, or consists of the amino acid sequence of SEQ ID NO: 180, or a sequence having at least 80%, at least 85%, at least 90%, at least 95%, at least 97%, at least 98%, at least 99%, or at least 99.5% identity to SEQ ID NO: 180. In embodiments, the AAV vector or AAV vector genome of the disclosure encodes OPA1 and is for treating Dominant Optic Atrophy.

In embodiments, the transgene encodes an Optic Atrophy 3 (OPA3). In embodiments, the OPA3 transgene comprises a mutant sequence, a codon-optimized sequence, and/or a truncated sequence of OPA3. In embodiments, the OPA3 transgene comprises, consists essentially of, or consists of a nucleic acid having the sequence of SEQ ID NO: 176, or a sequence having at least 80%, at least 85%, at least 90%, at least 95%, at least 97%, at least 98%, at least 99%, or at least 99.5% identity to SEQ ID NO: 176. In embodiments, the OPA3 transgene encodes a protein that comprises, consists essentially of, or consists of the amino acid sequence of SEQ ID NO: 181, or a sequence having at least 80%, at least 85%, at least 90%, at least 95%, at least 97%, at least 98%, at least 99%, or at least 99.5% identity to SEQ ID NO: 181. In embodiments, the AAV vector or AAV vector genome of the disclosure encodes OPA3 and is for treating Dominant Optic Atrophy.

In embodiments, the transgene comprises any one of the nucleic acid sequences listed in Table 4, or a sequence having up to 5, up to 10, or up to 30 nucleotide changes to any one of the DNA sequences in Table 4 (SEQ ID NOs: 116-118 and 172-176). In embodiments, the transgene encodes any one of the amino acid sequences listed in Table 4, or a sequence having up to 1, 2, 3, 4, 5, 6, 7, 8, 9, or 10 amino acids different from any one of the amino acid sequences listed in Table 4 (SEQ ID NOs: 142-144 and 177-181).

TABLE 4
Non-limiting Examples of Transgenes

| | | Amino acid SEQ | |
|---|---|---|---|
| | Nucleic acid | ID Nos. encoded | |
| Name of transgene | SEQ ID Nos. | by transgene | Features |
| Cytochrome P450 family | 116 | 142 | Natural |
| 4 subfamily V member 2 | | | |
| (CYP4V2) | | | |
| Retinoschisin 1 (RS1) | 117 | 143 | Natural |
| Phosphodiesterase 6B | 118 | 144 | Natural |
| (PDE6B) | | | |
| ATP binding cassette | 172 | 177 | Natural |
| subfamily A member 4 | | | |
| (ABCA4) | | | |
| Bestrophin-1 (BEST-1) | 173 | 178 | Natural |
| isoform 1 | | | |
| Bestrophin-1 (BEST-1) | 174 | 179 | Natural |
| isoform 2 | | | |
| OPA1 Mitochondrial | 175 | 180 | Natural |
| Dynamin Like GTPase | | | |
| (OPA1) | | | |
| Optic Atrophy 3 | 176 | 181 | Natural |
| (OPA3) | | | |

In embodiments, the transgene comprises a nucleic acid sequence set forth in any one of SEQ ID NOs: 99-133 and 172-176, or a sequence having up to 5, up to 10, or up to 30 nucleotide changes to any one of SEQ ID NOs: 99-133 and 172-176. In embodiments, the transgene encodes an amino acid sequence set forth in any one of SEQ ID NOs: 134-151 and 177-181, or a sequence having up to 1, 2, 3, 4, 5, 6, 7, 8, 9, or 10 amino acids different from any one of the amino acid sequences SEQ ID NOs: 134-151 and 177-181.

In embodiments, the heterologous nucleic acid encodes a reporter protein; for example, a fluorescent protein.

### Methods of Producing AAV Viral Vectors

A variety of approaches may be used to produce AAV viral vectors. In embodiments, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In embodiments, the cell is a packaging or helper cell line. In embodiments, the helper cell line is eukaryotic cell; for example, an HEK 293 cell or 293T cell. In embodiments, the helper cell is a yeast cell or an insect cell.

In embodiments, the cell comprises a nucleic acid encoding a tetracycline activator protein; and a promoter that regulates expression of the tetracycline activator protein. In embodiments, the promoter that regulates expression of the tetracycline activator protein is a constitutive promoter. In embodiments, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing AAV particles comprising transfecting a packaging cell line with any one of the AAV helper plasmids disclosed herein; and any one of the AAV vectors disclosed herein. In embodiments, the AAV helper plasmid and the AAV vector are co-transfected into the packaging cell line. In embodiments, the cell line is a mammalian cell line, for example, human embryonic kidney (HEK) 293 cell line. The disclosure provides cells comprising any one of the AAV vectors and/or AAV particles disclosed herein.

### Pharmaceutical Compositions

The disclosure provides pharmaceutical compositions comprising any one of the AAV vectors, AAV capsids and/or AAV particles described herein. Typically, the AAV particles are administered for therapy.

The pharmaceutical composition, as described herein, may be formulated by any methods known or developed in the art of pharmacology, which include but are not limited to contacting the active ingredients (e.g., viral particles or AAV vectors) with an excipient or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with AAV vectors or transduced with AAV viral particles (e.g., for transplantation into a subject), nanoparticle mimics or combinations thereof. In embodiments, the pharmaceutical composition is formulated as a nanoparticle. In embodiments, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients, each in an amount that together increases the stability of the viral vector, increases cell transfection or transduction by the viral vector, increases the expression of viral vector encoded protein, and/or alters the release profile of viral vector encoded proteins. In embodiments, the pharmaceutical composition comprises an excipient. Non limiting examples of excipients include solvents, dispersion media, diluents, or other liquid vehicles, dispersion or suspension aids, surface active agents, isotonic agents, thickening or emulsifying agents, preservatives, or combination thereof.

In embodiments, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

### Therapeutic Methods

This disclosure provides methods of preventing or treating a disorder, comprising, consisting essentially of, or consisting of administering to a subject a therapeutically effective amount of any one of the pharmaceutical compositions disclosed herein.

In embodiments, the disorder is a CNS disorder, a skin disorder, a lung disorder, a muscle disorder, a liver disorder, or an ophthalmic disease (or a retinal disease). In embodiments, the disorder is cystic fibrosis. In embodiments, the disorder is an ophthalmic disease. In embodiments, the disorder is a retinal disease.

In embodiments, the disorder is hypophosphatasia, amyotrophic lateral sclerosis (ALS), spinal muscular atrophy (SMA), recessive dystrophic epidermolysis bullosa (RDEB), lysosomal storage disorder (including Duchenne's Muscular Dystrophy, and Becker muscular dystrophy), juvenile Batten disease, infantile Batten disease, autosomal dominant disorders, muscular dystrophy, Bietti's Crystalline Dystrophy, retinoschisis (e.g., degenerative, hereditary, tractional, exudative), hemophilia A, hemophilia B, multiple sclerosis, diabetes mellitus, Fabry disease, Pompe disease, neuronal ceroid lipofuscinosis 1 (CLN1), CLN3 disease (or Juvenile Neuronal Ceroid Lipofuscinosis), Gaucher disease, cancer, arthritis, muscle wasting, heart disease, intimal hyperplasia, Rett syndrome, epilepsy, Huntington's disease, Parkinson's disease, Alzheimer's disease, an autoimmune disease, cystic fibrosis, thalassemia, Hurler's Syndrome (MPS IH), Sly syndrome, Scheie Syndrome, Hurler-Scheie Syndrome, Hunter's Syndrome, Sanfilippo Syndrome A (mucopolysaccharidosis IIIA or MPS IIIA), Sanfilippo Syndrome B (mucopolysaccharidosis IIIB or MPS IIIB), Sanfilippo Syndrome C, Sanfilippo Syndrome D, Morquio Syndrome, Maroteaux-Lamy Syndrome, Krabbe's disease, phenylketonuria, spinal cerebral ataxia, LDL receptor deficiency, hyperammonemia, anemia, arthritis, or adenosine deaminase deficiency.

In addition to specific transgenes disclosed herein, known active enzyme sequences may be used as transgenes to deliver functional enzyme activity.

In embodiments, the disorder is CLN3 disease. CLN3 disease or Juvenile Neuronal Ceroid Lipofuscinosis is a lysosomal storage disease caused by an autosomal recessively inherited mutation in the CLN3 gene. CLN3 disease is a progressive neurodegenerative disorder in which the central nervous system (CNS) is greatly affected resulting in behavioral issues, vision loss, and other cognitive disabilities.

In embodiments, the disorder is Fabry disease. Fabry disease is an X-linked lysosomal storage disorder caused by a deficiency in alpha-galactosidase A (GLA) activity that results in the accumulation of the glycolipid products, globotriaosylceramide (Gb3) and lyso-Gb3 in the lysosome. Disease presentation is highly heterogeneous but usually includes frequent bouts of peripheral neurotrophic pain, angiokeratomas, reduced sweat production, corneal dystrophy, and gastrointestinal complications. As the disease progresses patients suffer from cardiomyopathy, renal insufficiency and cerebrovascular disease, all of which are the primary causes of reduced life-span in Fabry patients. While males are the most severely affected population of patients with mutations in the GLA gene, it has become increasingly clear that female patients are also frequently symptomatic but are often misdiagnosed. Enzyme replacement therapy (ERT) is currently the only FDA-approved therapy to treat Fabry and requires bi-weekly injections of relatively large quantities of recombinant protein. While ERT reduces the accumulation of Gb3 in the heart, kidney and vasculature it fails to completely treat all symptoms of Fabry, primarily due to its inability to efficiently enter the CNS. Gene therapy strategies have been investigated and while many show great promise in correcting the glycolipid accumulation, most have failed to efficiently enter the CNS and also suffered from an immune response often seen during GLA replacement.

In embodiments, the AAV viral vectors disclosed herein are used to treat Fabry disease in patients, who are unresponsive to ERT, or when ERT fails to address all symptoms. In embodiments, the AAV viral vectors disclosed herein are used to treat Fabry disease in patients who have already been administered ERT.

In embodiments, the disorder is Pompe disease. Pompe disease is a lysosomal storage disorder caused by a deficiency in acid α-glucosidase (GAA) activity that results in the accumulation of glycogen in the lysosome. The disease presents as a form of muscular dystrophy which primarily affects both smooth and striated musculature as well as the central nervous system (CNS), with early mortality. Enzyme replacement therapy (ERT) is currently the only FDA-approved therapy to treat Pompe and requires bi-weekly injections of relatively large quantities of recombinant protein. While ERT significantly reduces the mortality rate of infantile Pompe patients, who typically die by the age of two without therapy, it fails to completely ameliorate all symptoms of Pompe, primarily due to its inability to efficiently enter the CNS and resulting immune responses to the GAA protein. Gene therapy strategies have been investigated and while many show great promise in correcting the glycogen accumulation and other symptoms of Pompe. Most have suffered from the severe immune response seen during GAA replacement. Previous work has demonstrated that hepatic-specific expression can make animals tolerate to the GAA protein and significantly reduce the humoral response.

In embodiments, the AAV viral vectors disclosed herein are used to treat Pompe disease in patients who have already been administered ERT; for example those who are unresponsive to ERT, or when ERT fails to address all their symptoms.

In embodiments, the AAV viral vectors disclosed herein are used to treat a cancer. In embodiments, the cancer is a solid cancer; for example, bladder, breast, cervical, colon, rectal, endometrial, kidney, lip, oral, liver, melanoma, mesothelioma, non-small cell lung, non-melanoma skin, ovarian, pancreatic, prostate, sarcoma, small cell lung tumor, or thyroid.

In embodiments, the disorder is an ophthalmic disease. The eye is immune privileged tissue. Only a very small number of viruses is necessary for therapeutic benefit. In embodiments, the ophthalmic disease affects photoreceptor and RPE cells. In embodiments, the ophthalmic disease comprises, consists essentially of, or consists of retinitis pigmentosa (e.g., autosomal recessive (SPATA7 gene; LRAT gene; TULP1 gene), autosomal dominant (AIPL1 gene), and X-linked (RPGR gene)), eye disorders related to mutations in the bestrophin-1 (BEST-1 or BEST1) gene (e.g., vitelliform macular dystrophy, age-related macular degeneration, autosomal dominant vitreoretinochoroidopathy, glaucoma, cataracts), Leber congenital amaurosis (LCA; aryl-hydrocarbon interacting protein-like 1 (AIPL1) gene), cone-rod dystrophy (CRD; ABCA4 gene), Stargardt's (ABCA4 gene), choroideremia (CHM gene), Usher Syndrome (MYO7A gene; CDH23 gene; USH2A gene; CLRN1 gene), Dominant Optic Atrophy (e.g., autosomal (OPA1 gene; OPA3 gene)), retinitis pigmentosa (PDE6B gene), retinoschisis (RS1 gene), Bietti's Crystalline Dystrophy (CYP4V2 gene) or Achromatopsia (CNGA3 gene, CNGB3 gene, GNAT2 gene, PDE6C gene, or PDE6H gene).

In embodiments, the disclosure provides methods of expressing a transgene in a retinal cell. In embodiments, the method comprises delivering a nucleic acid of the disclosure to the retinal cell. In embodiments, the method comprises transducing the retinal cell with the AAV viral vector of the disclosure.

In embodiments, the target cells of the disclosure comprise retinal cells. In embodiments, the retinal cells comprise a photoreceptor, a bipolar cell, a retinal ganglion cell, a horizontal cell, or an amacrine cell. In embodiments, the retinal cells comprise a retinal ganglion cell. In embodiments, the retinal cells comprise a bipolar cell. In embodiments, the retinal cells comprise a horizontal cell. In embodiments, the retinal cells comprise an amacrine cell. In embodiments, the retinal cells comprise a photoreceptor. In embodiments, the photoreceptor comprises a rod cell and/or a cone cell.

In embodiments, the target cells of the disclosure comprise, consist essentially of, or consist of photoreceptor cells.

In embodiments, the subject is a mammal; for example, a human. In particular aspects, the human is an infant human; for example, under 3 years old, 2 years old, or under 1 year old.

The methods of treatment and prevention disclosed herein may be combined with appropriate diagnostic techniques to identify and select patients for the therapy or prevention. For example, the method of treating or preventing a disorder disclosed herein may further comprise steps of performing a genetic test to identify a gene mutation or deletion related to the disorder in the subject. In embodiments, the method of treating or preventing a disorder comprises administering to a subject who has been previously identified as carrying a mutation related to the disorder, or as being at high risk for developing the disorder (for example, based on hereditary factors).

The disclosure provides methods of increasing the level of a protein in a host cell, comprising contacting the host cell with any one of the AAV particles disclosed herein, wherein the AAV particle comprises any one of the AAV vector genomes disclosed herein, comprising an HNA sequence encoding the protein. In embodiments, the protein is a therapeutic protein. In embodiments, the host cell is in vitro, in vivo, or ex vivo. In embodiments, the host cell is derived from a subject. In embodiments, the subject suffers from a disorder, which results in a reduced level and/or functionality of the protein, as compared to the level and/or functionality of the protein in a normal subject.

In embodiments, the level of the protein is increased to level of about 1×10⁻⁷ ng, about 3×10⁻⁷ ng, about 5×10⁻⁷ ng, about 7×10⁻⁷ ng, about 9×10⁻⁷ ng, about 1×10⁻⁶ ng, about 2×106 ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×10⁻⁶ ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

The disclosure provides methods of introducing a gene of interest to a cell in a subject comprising contacting the cell with an effective amount of any one of the AAV viral particles disclosed herein, wherein the AAV viral particle contains any one of the AAV vector genomes disclosed herein, comprising the gene of interest.

### Dosage and Administration

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10¹⁷ vector genomes per administration.

In embodiments of the methods described herein, the number of viral particles (e.g., AAV) administered to the subject ranges from about 10⁹ to about 10¹⁷. In embodiments, about 10¹⁰ to about 10¹², about 10¹¹ to about 10¹³, about 10¹¹ to about 10¹², about 10¹¹ to about 10¹⁴, about 5×10¹¹ to about 5×10¹², or about 10¹² to about 10¹³ viral particles are administered to the subject. For administration to a human eye, a total dose of about 1×10¹⁰ vg/eye may be used, and a total dose of 5×10⁹ vg/eye may be used for a mouse eye. Non-invasive, in vivo imaging techniques can be used to monitor efficacy/safety in animals, which include but are not limited to scanning laser ophthalmoscopy (SLO), optical coherence tomography (OCT), multi-photon microscopy, fluorescein angiography.

In embodiments, the AAV particles repair the gene deficiency in a subject. In embodiments, the ratio of repaired target polynucleotide or polypeptide to unrepaired target polynucleotide or polypeptide in a successfully treated cell, tissue, organ or subject is at least about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to Western analysis, Northern analysis, Southern analysis, PCR, sequencing, mass spectrometry, flow cytometry, immunohistochemistry, immunofluorescence, fluorescence in situ hybridization, next generation sequencing, immunoblot, and ELISA.

In embodiments, the viral particle is introduced to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intrapleurally, topically, intralymphatically, intracisternally; such introduction may also be intra-arterial, intracardiac, subventricular, epidural, intracerebral, intracerebroventricular, sub-retinal, para-retinal, intravitreal, intraarticular, intraperitoneal, intrauterine, or any combination thereof. In embodiments, the viral particles are delivered to a desired target tissue, e.g., to the lung, eye, or CNS, as non-limiting examples. In embodiments, delivery of viral particles is systemic. The intracisternal route of administration involves administration of a drug directly into the cerebrospinal fluid of the brain ventricles. It could be performed by direct injection into the cisterna magna or via a permanently positioned tube.

For treating an ophthalmic disease (or an eye disorder) intraocularly, there are multiple modes of administration known to those skilled in the art, including but not limited to: lacrimal gland (LG) administration, topical eye drop, intra-stromal administration to the cornea, intra-cameral administration (anterior chamber), intravitreal administration, sub-retinal administration, para-retinal administration, systemic administration, or a combination thereof. 80% of genetic eye disorders occur in the photoreceptors. Intravitreal delivery of small volume gene therapies can occur in an out-patient clinic.

In embodiments, the mode of administration is para-retinal administration. As used herein, the term “para-retinal administration” refers to a form of intravitreal administration that injects the therapeutic agent (e.g., an AAV viral vector) into the vitreous cavity in close proximity to the desired region of the retina (i.e., targeted delivery). In embodiments, the desired region of the retina is near the fovea area of the retina. In contrast to routine intravitreal administrations, which are done using short needles designed to deposit product in the mid-vitreous cavity, and do not require direct visualization, para-retinal injection is done under direct visualization of a longer needle capable of delivering product in the posterior vitreous cavity close to the retina. In embodiments, the therapeutic agent is deposited in the vitreous cavity at a distance of 0 mm to 13 mm from the surface of the retina, at a distance of 0 mm to 10 mm from the surface of the retina, at a distance of 0 mm to 5 mm from the surface of the retina, or at a distance of 0 mm to 3 mm from the surface of the retina. In embodiments, the therapeutic agent is deposited in the vitreous cavity at a distance of between 0-13 mm, between 0-12 mm, between 0-11 mm, between 0-10 mm, between 0-9 mm, between 0-8 mm, between 0-7 mm, between 0-6 mm, between 0-5 mm, between 0-4 mm, between 0-3 mm, between 0-2 mm, or between 0-1 mm, from the surface of the retina.

In embodiments, para-retinal administration is used in situations in which sub-retinal injection is not appropriate. In embodiments, para-retinal administration is used for targeted transduction of optic nerves. In embodiments, para-retinal administration is used for treating diseases or disorders that are related to dysfunction of the optic nerves. In embodiments, para-retinal administration is used for treating Dominant Optic Atrophy or Retinoschisis.

In embodiments, para-retinal administration involves the use of a small gauge needle (30 gauge or similar) with length sufficient to reach the posterior pole of the human eye (25 mm or similar), exo- or endo-illumination and visualization using a microscope, and use of a corneal contact lens to allow focus on the posterior vitreous cavity and retina. This is typically conducted after adequate analgesia and antisepsis, at which time the corneal contact lens is coupled to the eye and the microscope is positioned to view the posterior retina. The needle is inserted through the eye wall in the pars plana region and its tip is visualized. Under direct visualization, the needle tip is advanced to the desired location close to the retinal surface. The syringe plunger is advanced to slowly deposit the viral vector (which may be contained in any suitable composition or formulation). The needle is withdrawn and the eye is inspected. The port may be closed with a suture, but for a sufficiently small-caliber needle (such as 30 gauge), no suture is needed to close the needle tract. Ointment and an eye shield may be applied and, if desired, the subject can be kept in a supine position for a period post-operatively to further facilitate a high para-retinal concentration of the product. Variations on delivery instrumentation can include creation of a sclerotomy with or without the use of a vitrectomy port to allow use of a blunt cannula, and/or a cannula design with a tapered and/or flexible extendable tip or side ports to optimize proximity to the retinal surface and safety, and/or use of a pneumatic system in lieu of a simple syringe plunger. Additional descriptions of para-retinal administration are disclosed, for example, in WO 2020/018766 and Zeng et al., Mol Ther Methods Clin Dev. 2020 Sep. 11; 18: 422-427, the contents of each of which are incorporated herein by reference in their entireties for all purposes.

In embodiments, the mode of administration is sub-retinal administration, which injects the materials into the sub-retinal space between retinal pigment epithelium (RPE) cells and photoreceptors. In the sub-retinal space, the injected materials come into direct contact with the plasma membrane of the photoreceptor, and RPE cells and sub-retinal blebs. In embodiments, the AAV used for sub-retinal administration comprises the capsid protein of AAV214 or AAV214-D5. In embodiments, the sub-retinal administration is for treating Stargardt disease, Bietti's Crystalline Dystrophy, or BEST vitelliform macular dystrophy. Additional descriptions of sub-retinal administration are disclosed, for example, in Peng et. al., Ophthalmic Res 2017; 58:217-226; and Hartman et al., J Ocul Pharmacol Ther. 2018 Mar. 1; 34(1-2): 141-153, the contents of each of which are incorporated herein by reference in their entireties for all purposes.

Administration of the AAV viral particle or compositions of this disclosure can be effected in one dose, continuously or intermittently throughout the course of treatment. In embodiments, the AAV viral particle or compositions of this disclosure are parenterally administered by injection, infusion or implantation.

In embodiments, the AAV particles of this disclosure show enhanced tropism for brain and cervical spine. In embodiments, the viral particles of the disclosure can cross the blood-brain-barrier (BBB). In embodiments, the AAV particles of this disclosure show high retinal tropism by para-retinal, sub-retinal and/or intravitreal injections. In embodiments, the AAV particles of this disclosure target multiple eye cell types, such as, for example, cones, rods, and retinal pigment epithelium (RPE). In embodiments, AAV particles of this disclosure escape neutralizing antibodies against natural serotypes, and thus enable potential redosing. In a further aspect, the AAV particles and compositions of the disclosure may be administered in combination with other known treatments for the disorder being treated.

### Kits

The agents, viral vectors, or compositions described herein may, in embodiments, be assembled into pharmaceutical or diagnostic or research kits to facilitate their use in therapeutic, diagnostic or research applications. In embodiments, the kits of the present disclosure include any one of the modified AAV capsid proteins, AAV vectors, AAV viral particles, host cells, isolated tissues, compositions, or pharmaceutical compositions as described herein.

In embodiments, a kit further comprises instructions for use. Specifically, such kits may include one or more agents described herein, along with instructions describing the intended application and the proper use of these agents. As an example, in embodiments, the kit may include instructions for mixing one or more components of the kit and/or isolating and mixing a sample and applying to a subject. In embodiments, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In embodiments, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In embodiments, the preservation solution contains an amount of metalloprotease inhibitors.

In embodiments, the kit contains any one or more of the components described herein in one or more containers. Thus, in embodiments, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to a subject, such as a syringe, topical application devices, or IV needle tubing and bag.

It is to be understood that while the invention has been described in conjunction with the above embodiments, the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

### Further Numbered Embodiments

Embodiment 1. A method of treating an ophthalmic disease or disorder in a subject in need thereof, comprising para-retinal administration of an AAV viral vector to the subject, wherein the AAV viral vector comprises an AAV capsid protein comprising the amino acid sequence of SEQ ID NO: 2.

Embodiment 2. A method of treating an ophthalmic disease or disorder in a subject in need thereof, comprising para-retinal administration of an AAV viral vector to the subject, wherein the AAV viral vector comprises an AAV capsid protein comprising or consisting of an amino acid sequence that is at least 95%, at least 98%, at least 99%, at least 99.5%, or 100% identical to any one of SEQ ID NO: 1-3, 30-34, 49, 67, 84, and 164.

Embodiment 3. The method of Embodiment 2, wherein the AAV viral vector comprises an AAV capsid protein comprising an amino acid sequence that is up to 1, 2, 3, 4, 5, 6, 7, 8, 9, or 10 amino acids different from any one of SEQ ID NO: 1-3, 30-34, 49, 67, 84 and 164.

Embodiment 4. The method of Embodiment 2, wherein the AAV viral vector comprises an AAV capsid protein comprising or consisting of the amino acid sequence of SED ID NO: 2 or an amino acid sequence that is up to 1, 2, 3, 4, 5, 6, 7, 8, 9, or 10 amino acids different from SEQ ID NO: 2.

Embodiment 5. The method of Embodiment 4, wherein the AAV capsid protein comprises a leucine (L) at amino acid 129 of SEQ ID NO: 2, an asparagine (N) at amino acid 586 of SEQ ID NO: 2, and a glutamic acid (E) at amino acid 723 of SEQ ID NO: 2.

Embodiment 6. The method of Embodiment 2, wherein the AAV viral vector comprises an AAV capsid protein comprising or consisting of the amino acid sequence of SED ID NO: 1 or an amino acid sequence that is up to 2, 2, 3, 4, 5, 6, 7, 8, 9, or 10 amino acids different from SEQ ID NO: 1.

Embodiment 7. The method of Embodiment 6, wherein the AAV capsid protein comprises a leucine (L) at amino acid 129, a proline (P) at amino acid 148, a arginine (R) at amino acid 152, a serine (S) at amino acid 153, a threonine (T) at amino acid 158, a lysine (K) at amino acid 163, a arginine (R) at amino acid 169, a tryptophan (W) at amino acid 306, a phenylalanine (F) at amino acid 308, and a asparagine (N) at amino acid 319, wherein the amino acid positions are numbered with respect to SEQ ID NO: 1.

Embodiment 8. The method of Embodiment 2, wherein the AAV viral vector comprises an AAV capsid protein comprising or consisting of the amino acid sequence of SED ID NO: 164 or an amino acid sequence that is up to 1, 2, 3, 4, 5, 6, 7, 8, 9, or 10 amino acids different from SEQ ID NO: 164.

Embodiment 9. The method of Embodiment 2, wherein the AAV viral vector comprises an AAV capsid protein comprising or consisting of the amino acid sequence of SED ID NO: 67 or an amino acid sequence that is up to 1, 2, 3, 4, 5, 6, 7, 8, 9, or 10 amino acids different from SEQ ID NO: 67.

Embodiment 10. The method of Embodiment 2, wherein the AAV viral vector comprises an AAV capsid protein comprising or consisting of the amino acid sequence of SED ID NO: 3 or an amino acid sequence that is up to 2, 2, 3, 4, 5, 6, 7, 8, 9, or 10 amino acids different from SEQ ID NO: 3.

Embodiment 11. The method of any one of Embodiments 2-3 and 10, wherein the AAV capsid protein comprises a VP3 portion comprising variable regions (VR) I to IX wherein:

(a) VR-II comprises amino acid sequence
(SEQ ID NO: 54)
DNNGVK,
(b) VR-III comprises amino acid sequence
(SEQ ID NO: 55)
NDGS,
(c) VR-IV comprises amino acid sequence
(SEQ ID NO: 56)
INGSGQNQQT
or
(SEQ ID NO: 171)
QSTGGTAGTQQ,
(d) VR-V comprises amino acid sequence
(SEQ ID NO: 57)
RVSTTTGQNNNSNFAWTA,
(e) VR-VI comprises amino acid sequence
(SEQ ID NO: 58)
HKEGEDRFFPLSG,
(f) VR-VII comprises amino acid sequence
(SEQ ID NO: 59)
KQNAARDNADYSDV,
(g) VR-VIII comprises amino acid sequence
(SEQ ID NO: 60)
ADNLQQQNTAPQI,
and
(h) VR-IX comprises amino acid sequence
(SEQ ID NO: 61)
NYYKSTSVDF.

Embodiment 12. The method of Embodiment 11, wherein the VR-I region comprises SASTGAS (SEQ ID NO. 52), NSTSGGSS (SEQ ID NO. 53), SSTSGGSS (SEQ ID NO. 87), or NGTSGGST (SEQ ID NO: 170).

Embodiment 13. The method of any one of Embodiments 1-11, wherein the ophthalmic disease or disorder is selected from the group consisting of dominant optic atrophy, retinitis pigmentosa, macular degeneration, an eye disorder related to mutations in the bestrophin-1 (BEST-1) gene, Leber congenital amaurosis, cone-rod dystrophy, Stargardt disease, choroideremia, Usher Syndrome, retinoschisis, Bietti's Crystalline Dystrophy and Achromatopsia.

Embodiment 14. The method of Embodiment 13, wherein the retinitis pigmentosa is autosomal recessive, autosomal dominant, or X-linked.

Embodiment 15. The method of Embodiment 13, wherein the eye disorder related to mutations in the BEST-1 gene is vitelliform macular dystrophy, age-related macular degeneration, autosomal dominant vitreoretinochoroidopathy, glaucoma, or cataract.

Embodiment 16. The method of any one of Embodiments 1-15, wherein the AAV viral vector comprises an AAV vector genome encoding a gene selected from SPATA7, LRAT, TULP1, AIPL1, RPGR, AIPL1, ABCA4, CHM, MY07A, CDH23, USH2A, CLRN1, RS1, CYP4V2, CNGA3, CNGB3, GNAT2, RHO, PDE6B, PDE6C, PDE6H, OPA1, OPA3, and BEST-1.

Embodiment 17. The method of any one of Embodiments 1-15, wherein the AAV viral vector comprises an AAV vector encoding an antisense RNA, microRNA, siRNA, or guide RNA (gRNA).

Embodiment 18. The method of any one of Embodiments 1-17, wherein the ophthalmic disease or disorder is related to a dysfunction of optic nerve.

Embodiment 19. The method of any one of Embodiments 1-17, wherein the ophthalmic disease or disorder is Dominant Optic Atrophy.

Embodiment 20. The method of Embodiment 19, wherein the AAV viral vector comprises an AAV vector genome comprising an OPA1 or OPA3 transgene.

Embodiment 21. The method of any one of Embodiments 1-17, wherein the ophthalmic disease or disorder is Retinoschisis.

Embodiment 22. The method of Embodiment 21, wherein the AAV viral vector comprises an AAV vector genome comprising a RS1 transgene.

Embodiment 23. The method of any one of Embodiments 1-22, wherein the para-retinal administration comprises injecting at a distance of between 0-13 millimeters (mm), between 0-10 mm, between 0-5 mm, or between 0-3 mm, from the surface of the retina in the posterior vitreous cavity of the eye.

Embodiment 23.1. The method of any one of Embodiments 1-22, wherein the para-retinal administration comprises injecting at a distance of between 0-13 mm from the surface of the retina in the posterior vitreous cavity of the eye.

Embodiment 23.2. The method of any one of Embodiments 1-22, wherein the para-retinal administration comprises injecting at a distance of between 0-10 mm from the surface of the retina in the posterior vitreous cavity of the eye.

Embodiment 23.3. The method of any one of Embodiments 1-22, wherein the para-retinal administration comprises injecting at a distance of between 0-5 mm from the surface of the retina in the posterior vitreous cavity of the eye.

Embodiment 23.4. The method of any one of Embodiments 1-22, wherein the para-retinal administration comprises injecting at a distance of between 0-3 mm from the surface of the retina in the posterior vitreous cavity of the eye.

Embodiment 24. The method of any one of Embodiments 1-23.4, wherein the subject is a human.

Embodiment 25. A nucleic acid encoding an AAV capsid protein comprising a VP3 portion, wherein the VP3 portion comprises variable regions (VR) I to IX wherein:

(a) VR-II comprises amino acid sequence
(SEQ ID NO: 54)
DNNGVK,
(b) VR-III comprises amino acid sequence
(SEQ ID NO: 55)
NDGS,
(c) VR-IV comprises amino acid sequence
(SEQ ID NO: 171)
QSTGGTAGTQQ,
(d) VR-V comprises amino acid sequence
(SEQ ID NO: 57)
RVSTTTGQNNNSNFAWTA,
(e) VR-VI comprises amino acid sequence
(SEQ ID NO: 58)
HKEGEDRFFPLSG,
(f) VR-VII comprises amino acid sequence
(SEQ ID NO: 59)
KQNAARDNADYSDV,
(g) VR-VIII comprises amino acid sequence
(SEQ ID NO: 60)
ADNLQQQNTAPQI,
and
(h) VR-IX comprises amino acid sequence
(SEQ ID NO: 61)
NYYKSTSVDF.

Embodiment 26. The nucleic acid of Embodiment 25, wherein the VR-I region comprises NGTSGGST (SEQ ID NO: 170).

Embodiment 27. The nucleic acid of Embodiment 25, wherein the VP3 portion has the amino acid sequence of SEQ ID NO: 166.

Embodiment 28. The nucleic acid of any one of Embodiments 25-27, wherein the AAV capsid protein further comprises i) a VP2 portion or ii) a VP1 portion and a VP2 portion.

Embodiment 29. The nucleic acid of any one of Embodiments 25-27, wherein the encoded AAV capsid protein comprises an amino acid sequence at least 95%, at least 96%, at least 97%, at least 98%, at least 99%, or 100% identical to SEQ ID NO: 164 or an amino acid sequence having up to 1, 2, 3, 4, 5, 6, 7, 8, 9, or 10 amino acids different from SEQ ID NO: 164.

Embodiment 30. The nucleic acid of any one of Embodiments 25-27, wherein the encoded AAV capsid protein comprises an amino acid sequence at least 95%, at least 96%, at least 97%, at least 98%, at least 99%, or 100% identical to SEQ ID NO: 165 or an amino acid sequence having up to 1, 2, 3, 4, 5, 6, 7, 8, 9, or 10 amino acids different from SEQ ID NO: 165.

Embodiment 31. The nucleic acid of any one of Embodiments 25-27, wherein the encoded AAV capsid protein comprises an amino acid sequence at least 95%, at least 96%, at least 97%, at least 98%, at least 99%, or 100% identical to SEQ ID NO: 166 or an amino acid sequence having up to 1, 2, 3, 4, 5, 6, 7, 8, 9, or 10 amino acids different from SEQ ID NO: 166.

Embodiment 32. The nucleic acid of any one of Embodiments 25-31, wherein the nucleic acid sequence is at least 95% identical to the nucleotide sequence selected from SEQ ID NOs: 167-169.

Embodiment 33. The nucleic acid of any one of Embodiments 25-31, wherein the nucleic acid sequence is 100% identical to the nucleotide sequence selected from SEQ ID NOs: 167-169.

Embodiment 34. A vector comprising the nucleic acid of any one of Embodiments 25-33.

Embodiment 35. An AAV capsid protein encoded by the nucleic acid of any one of Embodiments 25-33.

Embodiment 36. The AAV capsid protein of Embodiment 35, wherein the protein comprises the amino acid sequence of SEQ ID NO: 164, 165 or 166, or an amino acid sequence having up to 1, 2, 3, 4, 5, 6, 7, 8, 9, or 10 amino acids different from SEQ ID NO: 164, 165 or 166.

Embodiment 37. An AAV viral vector comprising the AAV capsid protein encoded by the nucleic acid of any one of Embodiments 25-33 and an AAV vector genome, wherein the AAV vector genome comprises, in 5′ to 3′ orientation:
(a) a first AAV inverted terminal repeat,
(b) a promoter,
(c) a heterologous nucleic acid,
(d) a polyadenylation signal, and
(e) a second AAV inverted terminal repeat.

Embodiment 38. The AAV viral vector of Embodiment 37, wherein the heterologous nucleic acid is operably linked to a constitutive promoter.

Embodiment 39. The AAV viral vector of Embodiment 37 or 38, wherein the heterologous nucleic acid encodes a polypeptide.

Embodiment 40. The AAV viral vector of Embodiment 37 or 38, wherein the heterologous nucleic acid encodes an antisense RNA, an siRNA, a microRNA, or a gRNA.

Embodiment 41. The AAV viral vector of any one of Embodiments 37-41, wherein the AAV capsid protein comprises the amino acid sequence of SEQ ID NO: 164, 165 or 166.

Embodiment 42. An AAV viral vector comprising
(i) an AAV capsid protein having the amino acid sequence of SEQ ID NO: 164 and
(ii) an AAV vector genome, wherein the AAV vector genome comprises, in 5′ to 3′ orientation:
   (a) a first AAV inverted terminal repeat,
   (b) a promoter,
   (c) a heterologous nucleic acid,
   (d) a polyadenylation signal; and
   (e) a second AAV inverted terminal repeat.

Embodiment 43. The AAV viral vector of any one of Embodiments 37-39 and 41-42, wherein the heterologous nucleic acid encodes a polypeptide having at least 90% identity to any one of SEQ ID NOs: 142-144 and 177-181.

Embodiment 44. The AAV viral vector of Embodiment 43, wherein the heterologous nucleic acid comprises a polynucleotide sequence having at least 90% identity to any one of SEQ ID NOs: 116-118 and 172-176.

Embodiment 45. A method of treating a disease or disorder comprising administering the AAV viral vector of any of Embodiments 37-44 to a subject.

Embodiment 46. The method of Embodiment 45, wherein the AAV viral vector is administered to the subject orally, rectally, transmucosally, inhalationally, transdermally, parenterally, intravenously, subcutaneously, intradermally, intramuscularly, intrapleurally, intracerebrally, intrathecally, intracerebrally, intraventricularly, intranasally, intra-aurally, intra-ocularly, peri-ocularly, topically, intralymphatically, intracistemally, intravitreally, para-retinally, or sub-retinally.

Embodiment 47. The method of Embodiment 45 or 46, wherein the disease or disorder is amyotrophic lateral sclerosis (ALS), spinal muscular atrophy (SMA), Fabry disease, Pompe disease, CLN3 disease (or Juvenile Neuronal Ceroid Lipofuscinosis), recessive dystrophic epidermolysis bullosa (RDEB), juvenile Batten disease, autosomal dominant disorder, muscular dystrophy, hemophilia A, hemophilia B, multiple sclerosis, diabetes mellitus, Gaucher disease cancer, arthritis, muscle wasting, heart disease, intimal hyperplasia, epilepsy, Huntington's disease, Parkinson's disease, Alzheimer's disease, cystic fibrosis, thalassemia, Hurler's Syndrome, Sly syndrome, Scheie Syndrome, Hurler-Scheie Syndrome, Hunter's Syndrome, Sanfilippo Syndrome A (mucopolysaccharidosis IIIA or MPS IIIA), Sanfilippo Syndrome B (mucopolysaccharidosis IIIB or MPS IIIB), Sanfilippo Syndrome C, Sanfilippo Syndrome D, Morquio Syndrome, Maroteaux-Lamy Syndrome, Krabbe's disease, phenylketonuria, Batten's disease, spinal cerebral ataxia, LDL receptor deficiency, hyperammonemia, arthritis, macular degeneration, retinitis pigmentosa, ceroid lipofuscinosis, neuronal, 1 (CLN1), adenosine deaminase deficiency, Dominant Optic Atrophy, Retinoschisis, Stargardt disease, Bietti's Crystalline Dystrophy or BEST vitelliform macular dystrophy.

Embodiment 48. The method of Embodiment 45 or 46, wherein the diseases or disorder is an ophthalmic disease or disorder.

Embodiment 49. The method of Embodiment 48, wherein the ophthalmic disease or disorder is selected from the group consisting of dominant optic atrophy, retinitis pigmentosa, macular degeneration, an eye disorder related to mutations in the bestrophin-1 (BEST-1) gene, Leber congenital amaurosis, cone-rod dystrophy, Stargardt disease, choroideremia, Usher Syndrome, retinoschisis, Bietti's Crystalline Dystrophy and Achromatopsia.

Embodiment 50. The method of any of Embodiments 45-49, wherein the subject is a human.

### EXAMPLES

### Example 1

### Characterization of Non-Human Primate Para-Retinal and Sub-Retinal Dosing Using Various AAV Viral Vectors

The transduction efficiency of the AAV viral vectors comprising an AAV204, AAV214, AAV214-D5, or AAV8 capsid via multiple ocular administration modes was assessed as described below.

All AAV viral vectors used in this example comprises a recombinant nucleic acid encoding an enhanced green fluorescence protein (“EGFP” or “GFP” hereinafter) reporter transgene that is operably linked to the CBh promoter.

To test transduction efficiency of the AAV viral vectors in vivo, non-human primates (NHP) Macaca fascicularis were dosed by para-retinal, sub-retinal, or intravitreal administration of the indicated AAV viral vectors. The dose/volume for each administration mode were:
Para-retinal dosing—1.0E+11 vg in 100 μL injection volume
Sub-retinal dosing—2.5E+10 vg in 100 μL injection volume
Intravitreal dosing—1.5E+12 vg in 150 μL injection volume
   Para-retinal administration was performed by layering virus on top of the retina between the vitreous and the inner limiting membrane, thus not creating a subretinal detachment. GFP expression was monitored using scanning laser ophthalmoscopy (SLO). SLO images were taken on samples collected 26-27 days post injection. At 28 days post-injection, eyes were collected, processed, and analyzed by immunohistochemistry.

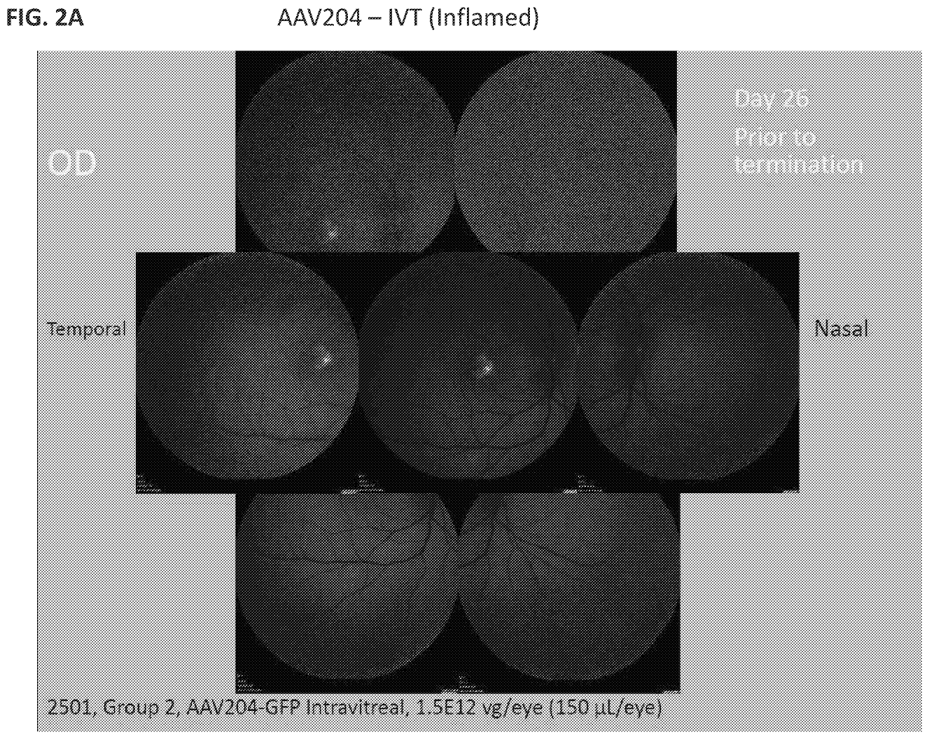

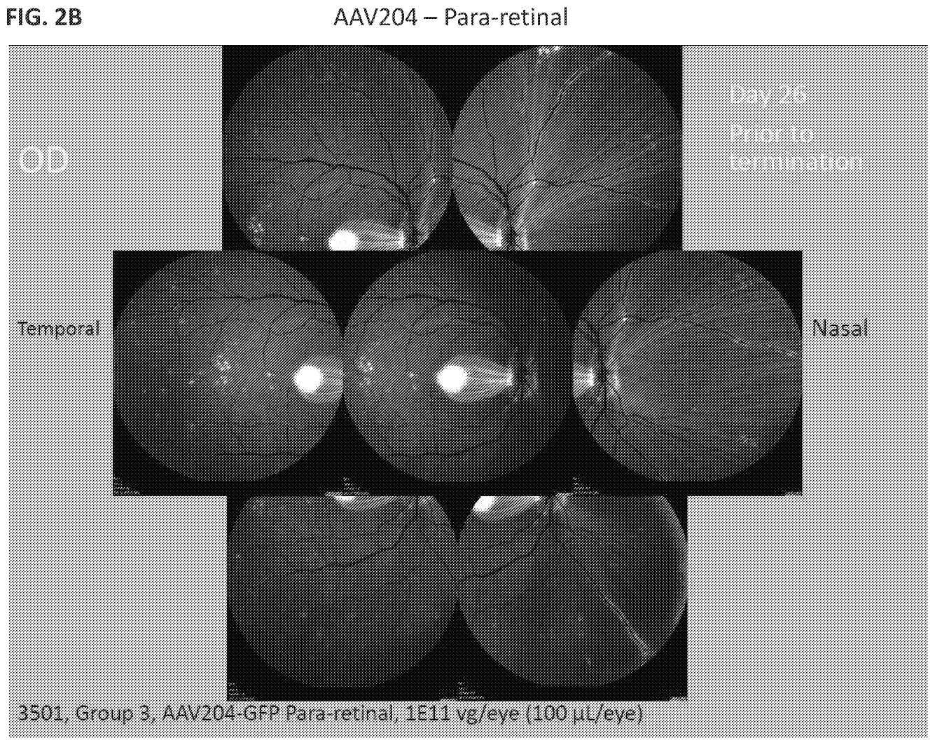

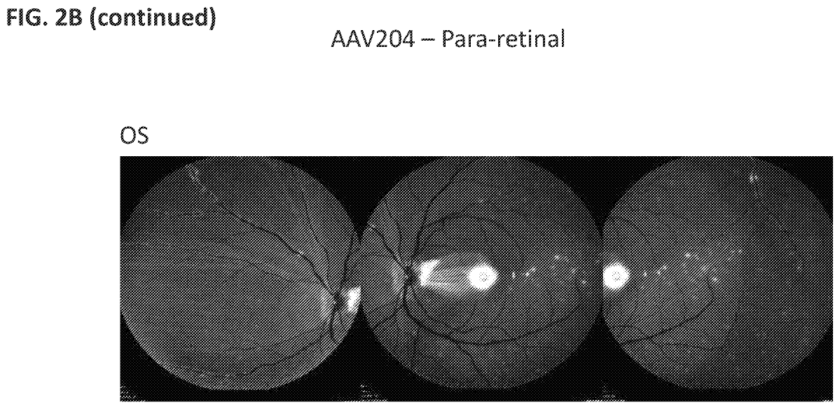

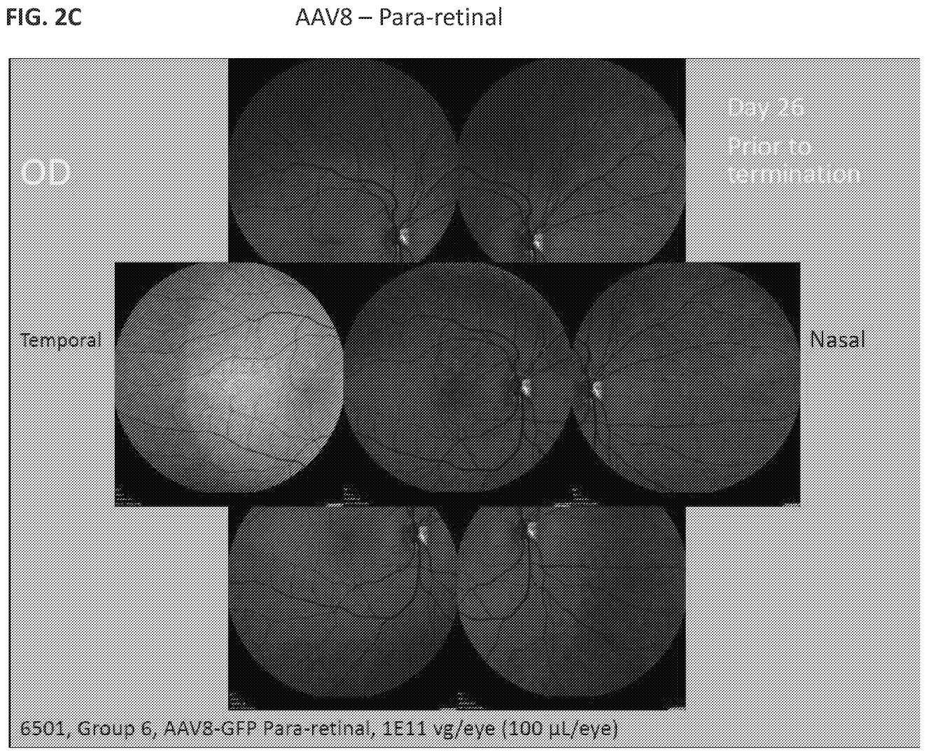

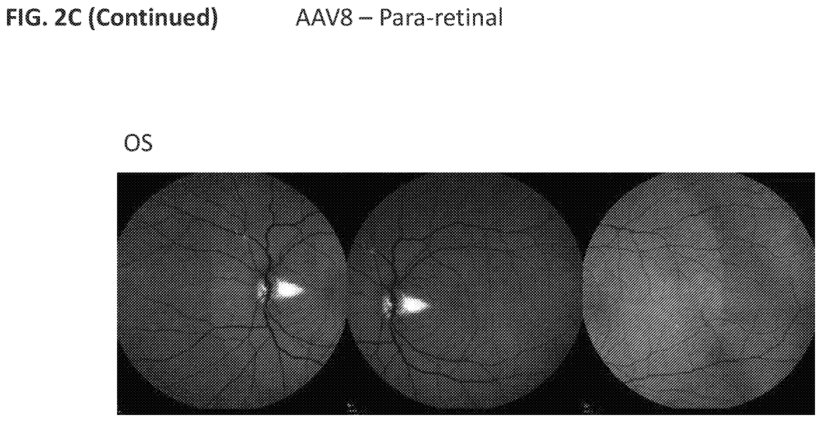

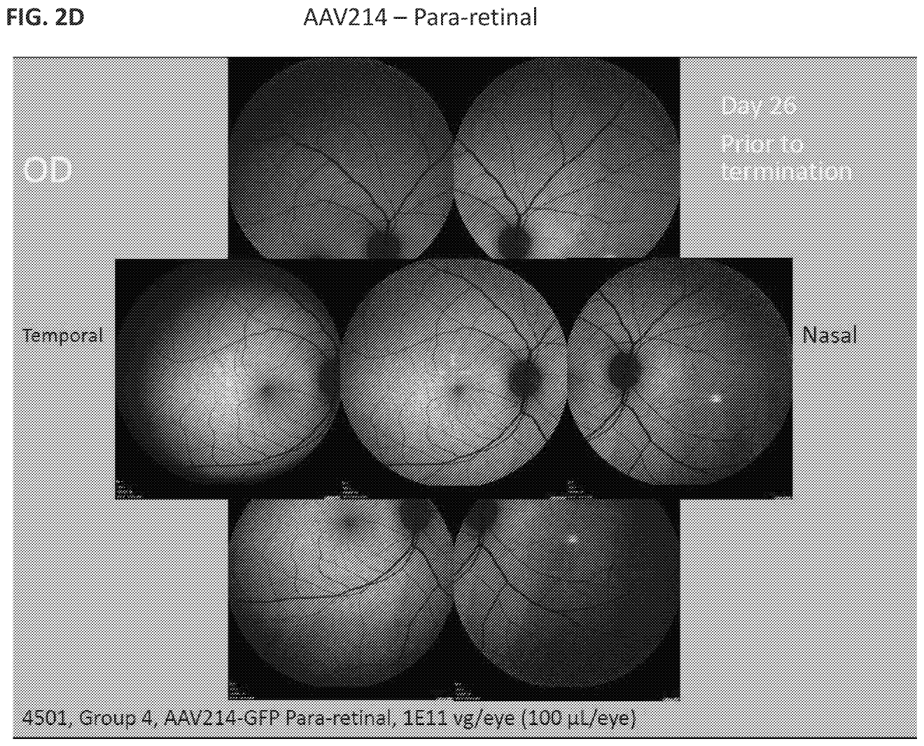

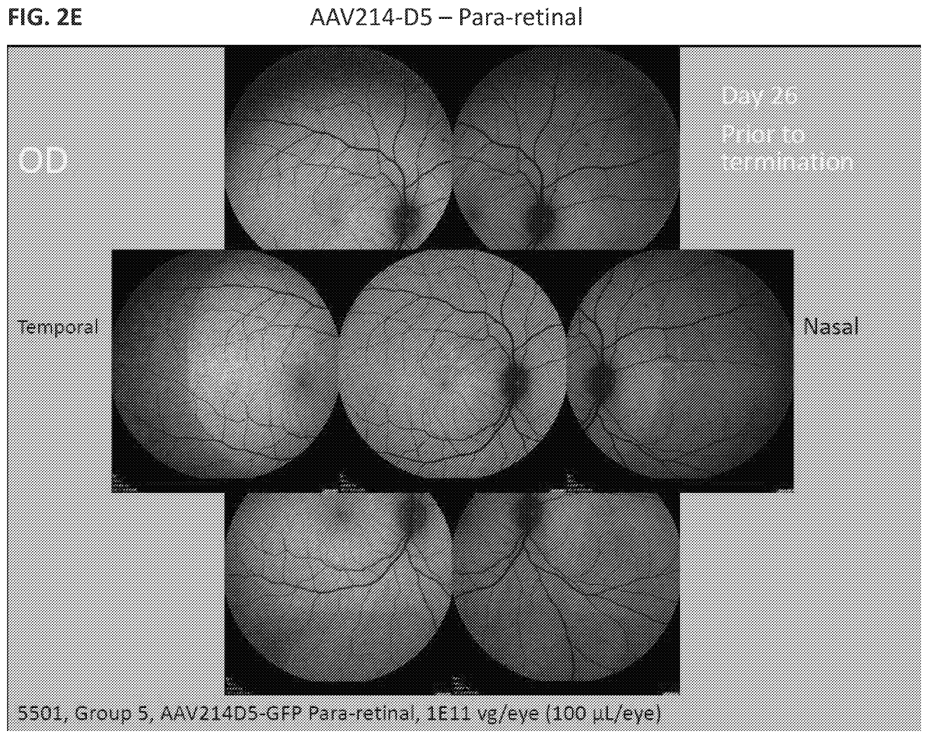

FIGS. 2B-2E shows the SLO results of para-retinal injection of the AAV viral vectors comprising the capsid protein of AAV204 (FIG. 2B), AAV8 (FIG. 2C), AAV214 (FIG. 2D), or AAV214-D5 (FIG. 2E). Among the capsid proteins tested, the AAV viral vector comprising AAV204 capsid demonstrates robust transduction in the macular, papillomacular bundle, and retinal nerve fibers via para-retinal injection, with a much higher transduction efficiency compared to other tested capsids.

In addition, the para-retinal administration of the AAV viral vector comprising AAV204 capsid (FIG. 2B) also shows much higher local transduction efficiency (particularly for optical nerve transduction) compared to the traditional intravitreal administration of the same AAV viral vector (FIG. 2A).

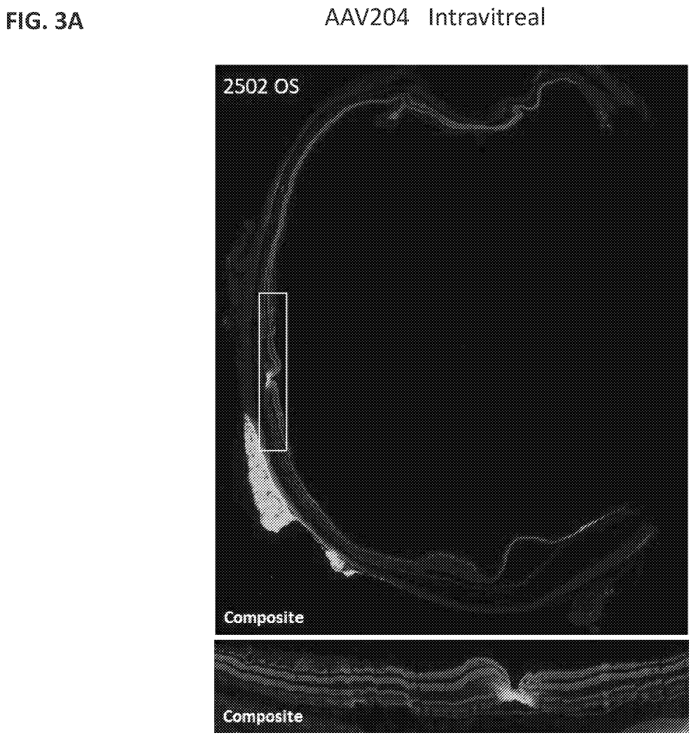

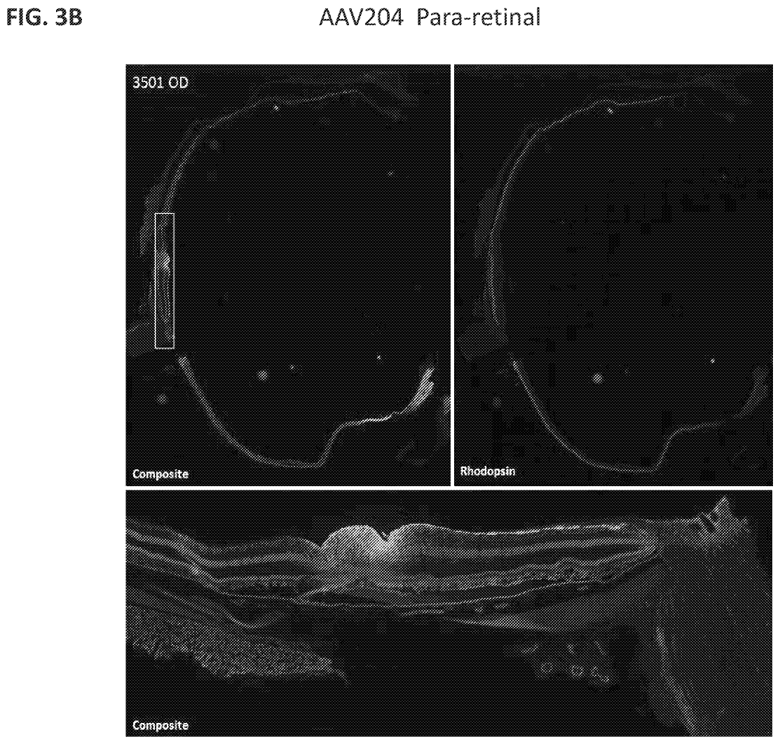

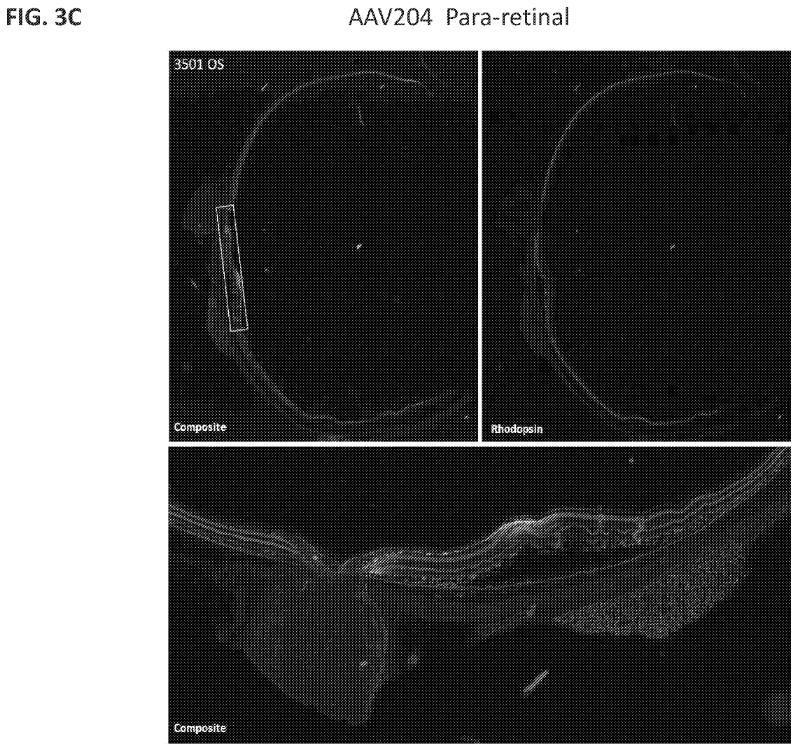

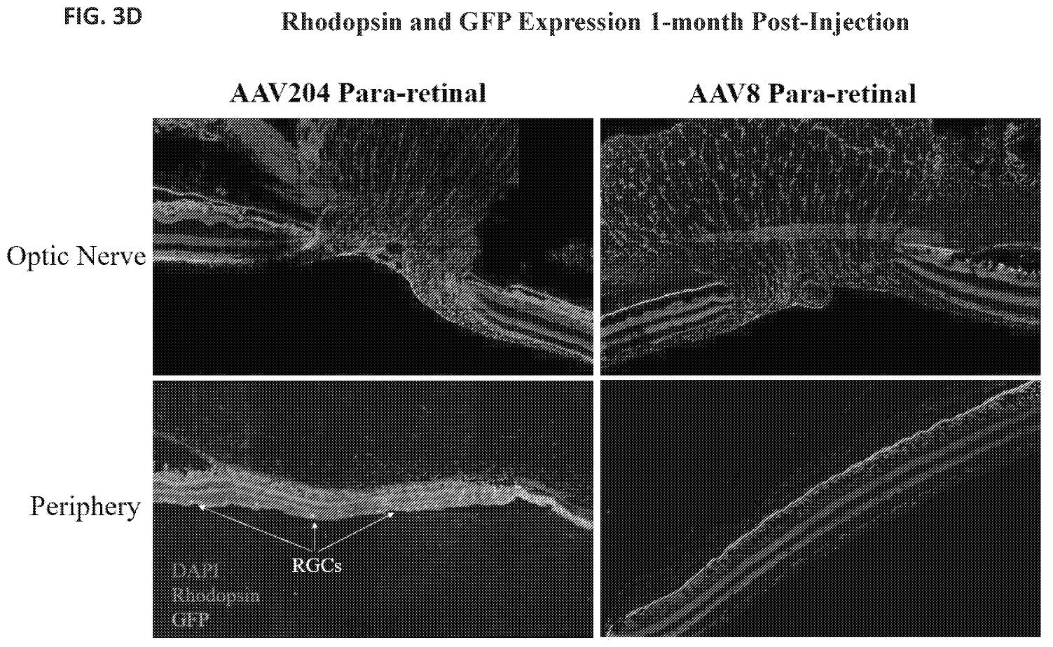

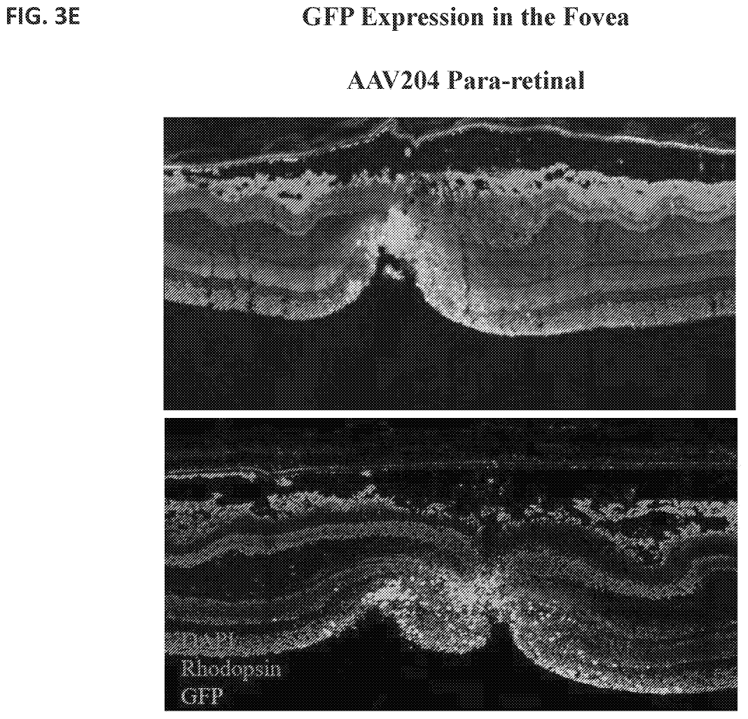

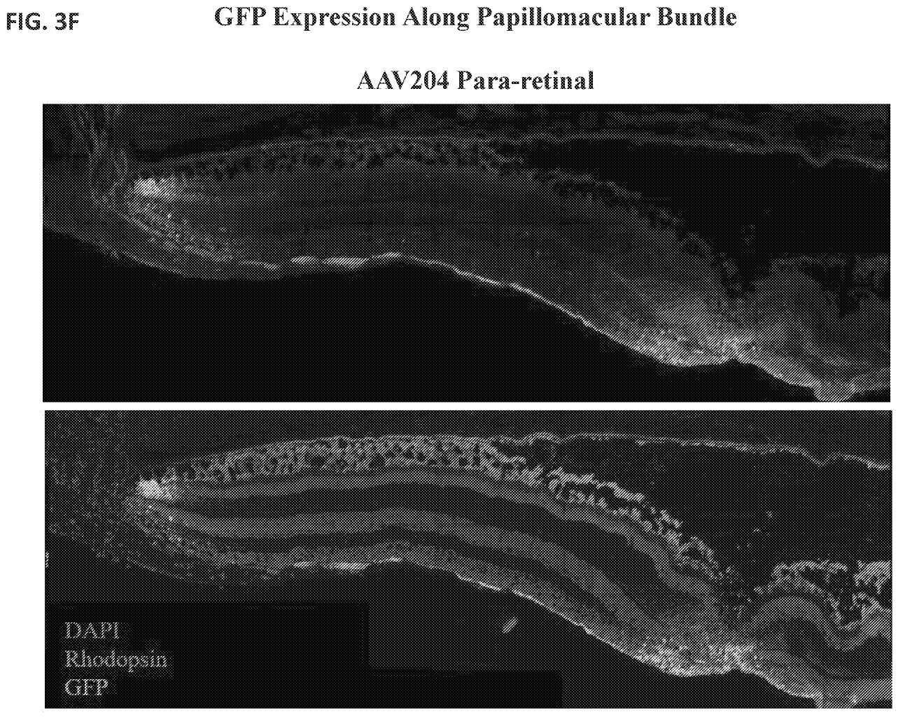

To further compare the transduction efficiency of these two administration routes, the retinas were processed for imaging analysis (FIGS. 3A-3C). Again, retinas receiving para-retinal administration of the AAV viral vector comprising AAV204 capsid (FIGS. 3B-3C) demonstrates much more robust macular and optic nerve transduction than the retinas receiving intravitreal administration of the same AAV viral vector (FIG. 3A).

Further immunohistochemistry analysis of rhodopsin and GFP 1-month post para-retinal injection of AAV204 viral vectors (FIG. 3D) showed high GFP expression in retinal ganglion cells (RGCs) across the entire retina and nerve fibers with high GFP expression were observed along the retina and entering the optic nerve. In comparison, para-retinal injection of AAV8 viral vectors (FIG. 3D) showed much lower GFP expression in RGCs. As shown in FIGS. 3E-3F, para-retinal administration of AAV204 viral vectors also resulted in robust GFP expression in NHP fovea and along the papillomacular bundle between the macula and optic nerve. These results were consistent with the SLO analysis. Thus, para-retinal administration of AAV204 viral vector results in efficient transduction of target cells in the macula and foveal pit as well as retinal ganglion cells and the associated retinal nerve fibers extending to the optic nerve, at a dose that is at least 10-fold lower compared to intravitreal AAV injections commonly used in the field.

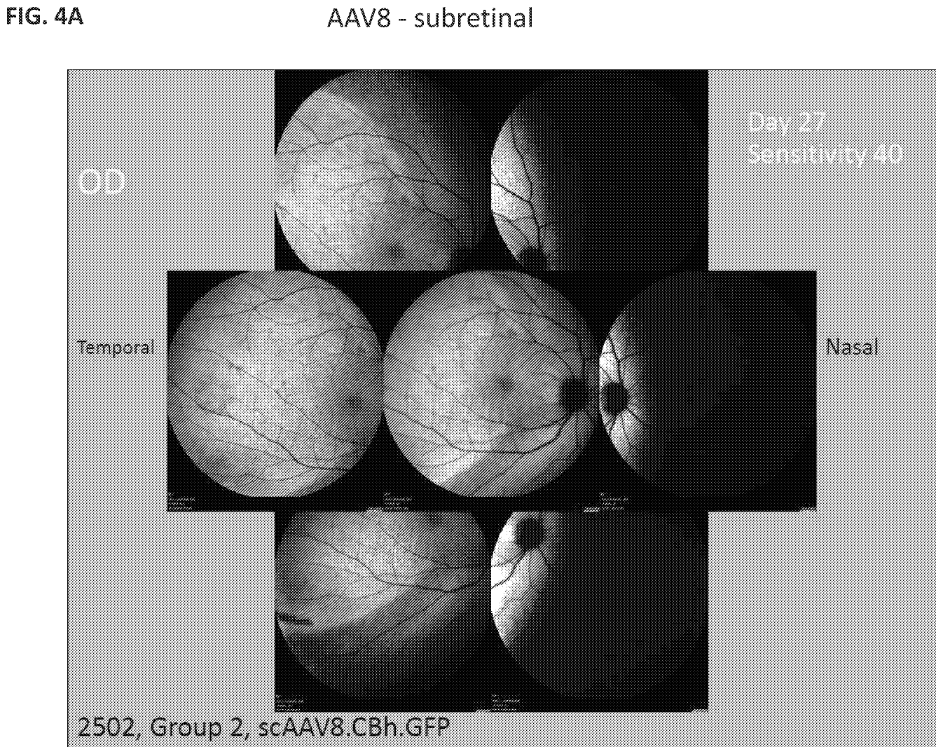

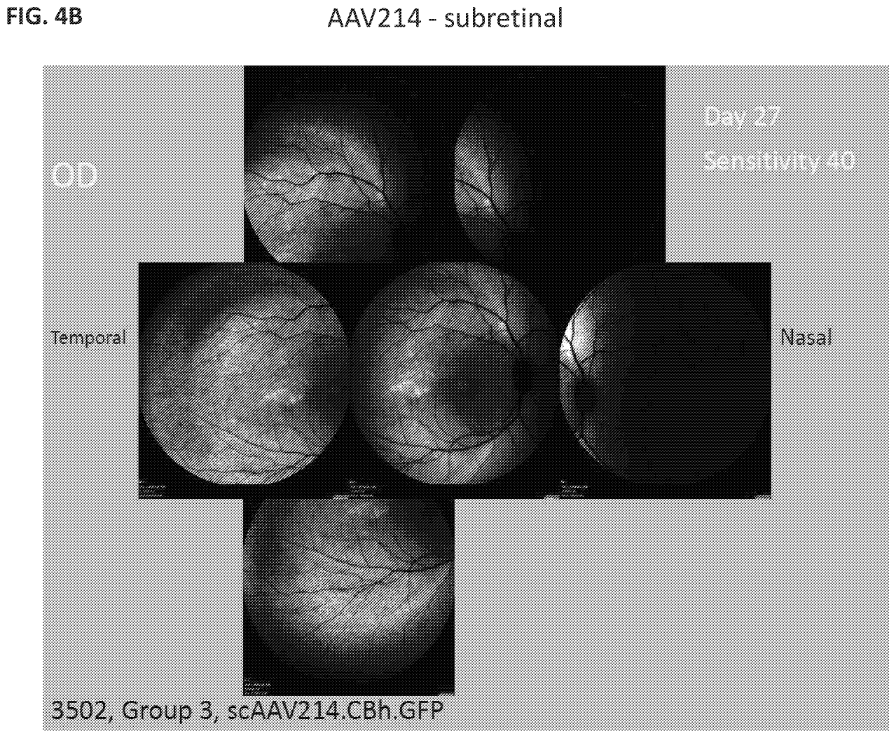

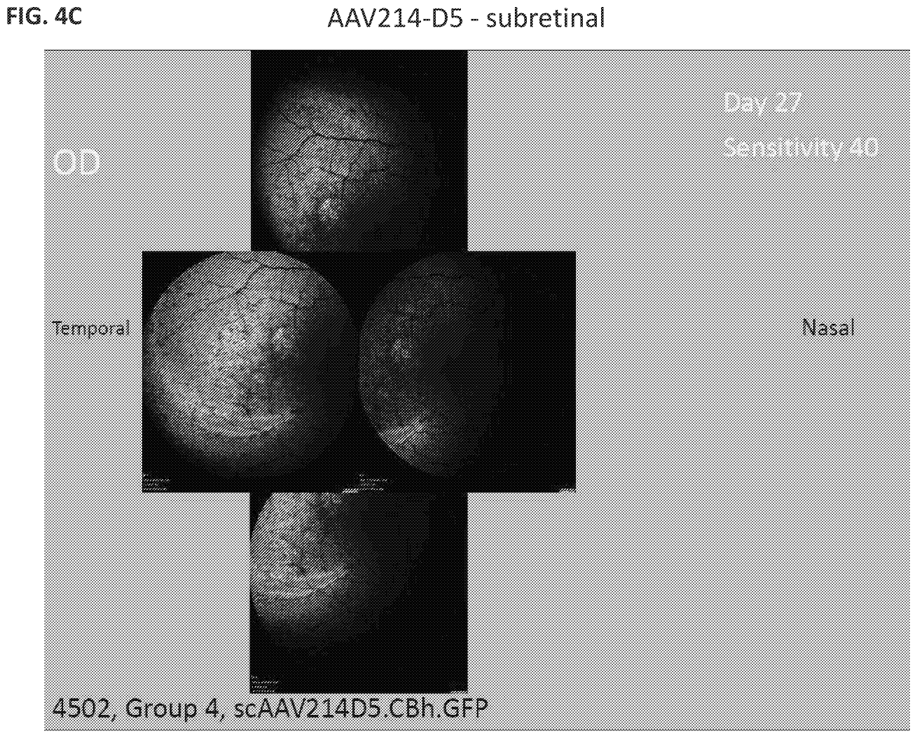

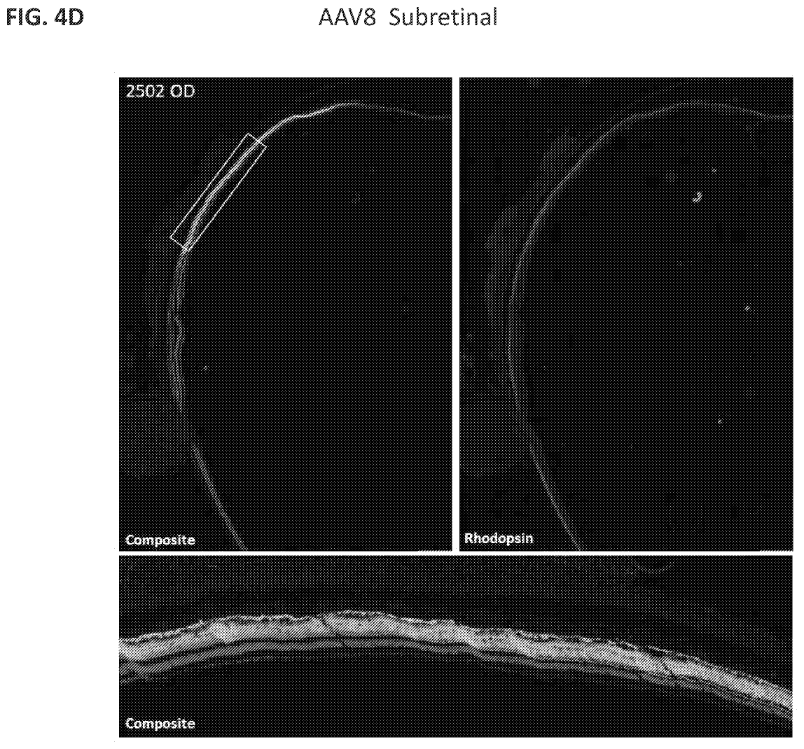

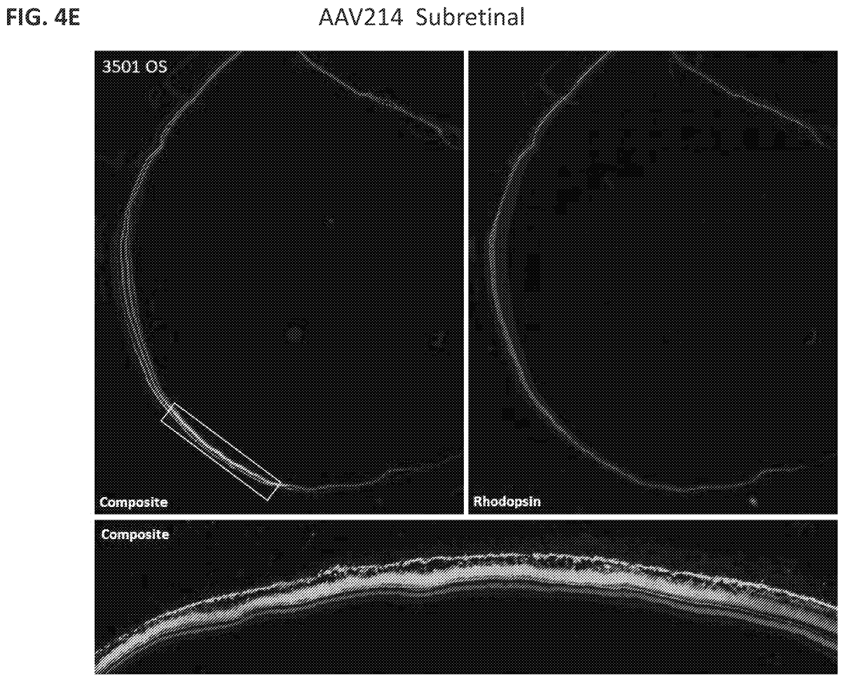

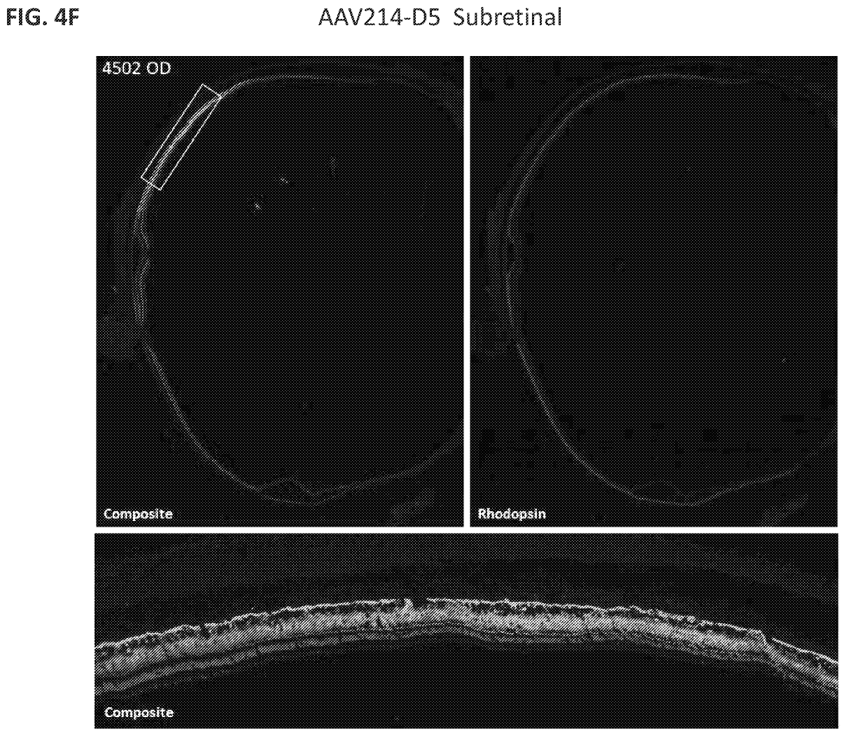

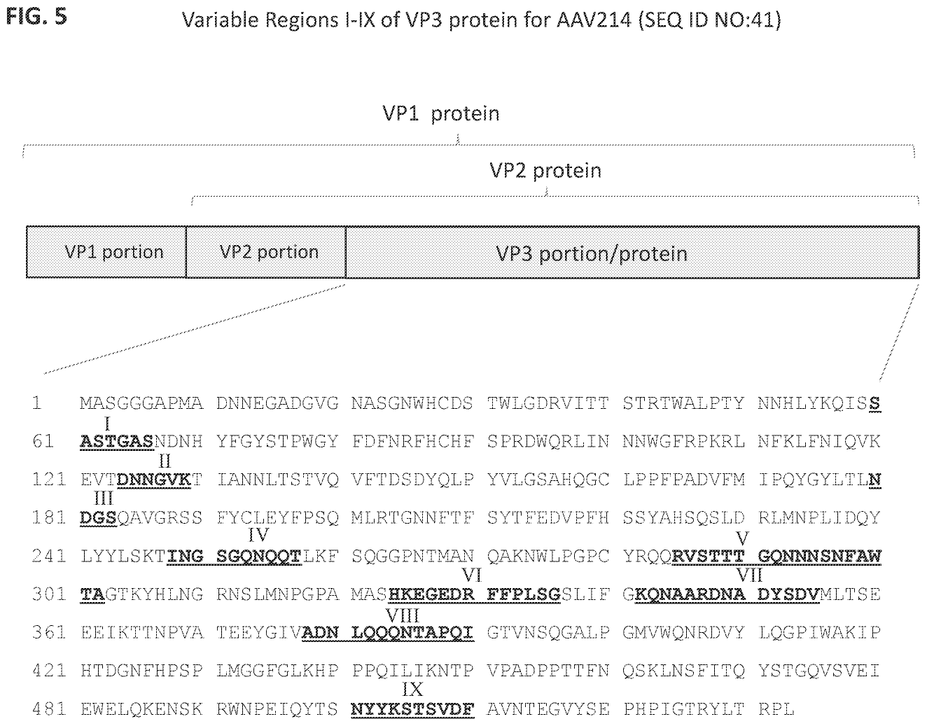

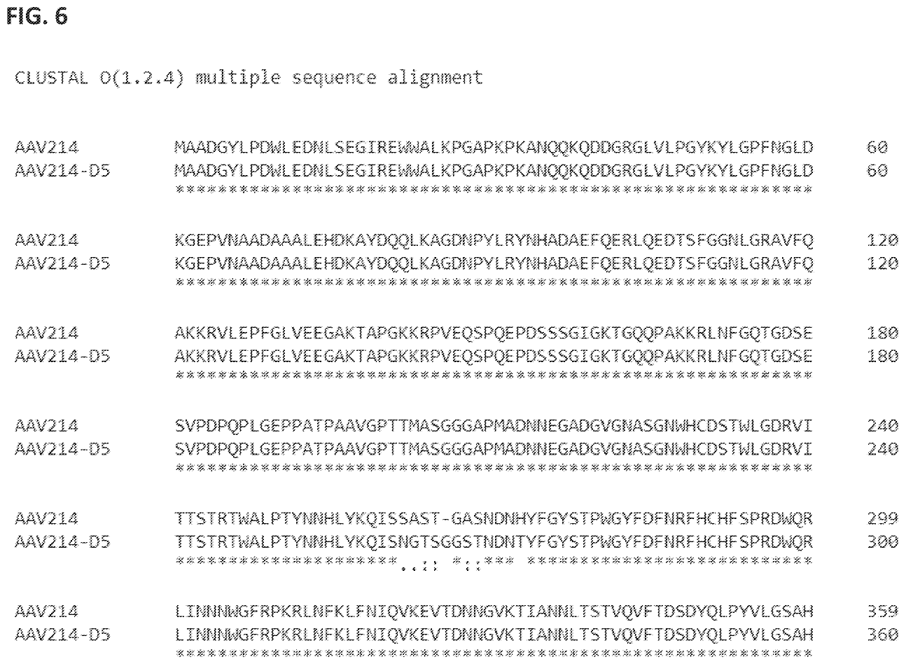

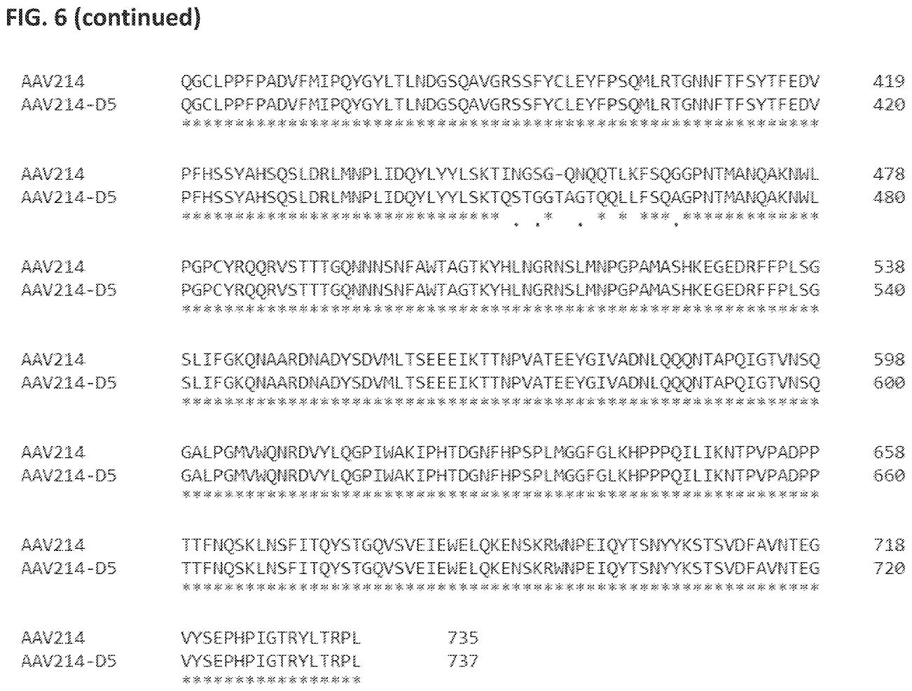

The transduction efficiency of the AAV viral vectors comprising the capsid protein of AAV8 (FIG. 4A; FIG. 4D), AAV214 (FIG. 4B; FIG. 4E), and AAV214-D5 (FIG. 4C; FIG. 4F) was also accessed. The results show that these 3 capsids display similar transduction efficiency when administered sub-retinally.

Conclusions: These results show that para-retinal injection of AAV vectors comprising AAV204 capsid can efficiently deliver the payload to the macula or optic nerve/retinal ganglion cell layer.

What is claimed is:

1. A method of treating an ophthalmic disease or disorder in a subject in need thereof, comprising para-retinal administration of an AAV viral vector to the subject, wherein the AAV viral vector comprises an AAV capsid protein comprising the amino acid sequence of SEQ ID NO: 2.

2. A method of treating an ophthalmic disease or disorder in a subject in need thereof, comprising para-retinal administration of an AAV viral vector to the subject, wherein the AAV viral vector comprises an AAV capsid protein comprising or consisting of an amino acid sequence that is at least 95%, at least 98%, at least 99%, at least 99.5%, or 100% identical to any one of SEQ ID NO: 1-3, 30-34, 49, 67, 84, and 164.

3. The method of claim 2, wherein the AAV viral vector comprises an AAV capsid protein comprising an amino acid sequence that is up to 1, 2, 3, 4, 5, 6, 7, 8, 9, or 10 amino acids different from any one of SEQ ID NO: 1-3, 30-34, 49, 67, 84 and 164.

4. The method of claim 2, wherein the AAV viral vector comprises an AAV capsid protein comprising or consisting of the amino acid sequence of SED ID NO: 2 or an amino acid sequence that is up to 1, 2, 3, 4, 5, 6, 7, 8, 9, or 10 amino acids different from SEQ ID NO: 2.

5. The method of claim 4, wherein the AAV capsid protein comprises a leucine (L) at amino acid 129 of SEQ ID NO: 2, an asparagine (N) at amino acid 586 of SEQ ID NO: 2, and a glutamic acid (E) at amino acid 723 of SEQ ID NO: 2.

6. The method of claim 2, wherein the AAV viral vector comprises an AAV capsid protein comprising or consisting of the amino acid sequence of SED ID NO: 1 or an amino acid sequence that is up to 2, 2, 3, 4, 5, 6, 7, 8, 9, or 10 amino acids different from SEQ ID NO: 1.

7. The method of claim 6, wherein the AAV capsid protein comprises a leucine (L) at amino acid 129, a proline (P) at amino acid 148, a arginine (R) at amino acid 152, a serine (S) at amino acid 153, a threonine (T) at amino acid 158, a lysine (K) at amino acid 163, a arginine (R) at amino acid 169, a tryptophan (W) at amino acid 306, a phenylalanine (F) at amino acid 308, and a asparagine (N) at amino acid 319, wherein the amino acid positions are numbered with respect to SEQ ID NO: 1.

8. The method of claim 2, wherein the AAV viral vector comprises an AAV capsid protein comprising or consisting of the amino acid sequence of SED ID NO: 164 or an amino acid sequence that is up to 1, 2, 3, 4, 5, 6, 7, 8, 9, or 10 amino acids different from SEQ ID NO: 164.

9. The method of claim 2, wherein the AAV viral vector comprises an AAV capsid protein comprising or consisting of the amino acid sequence of SED ID NO: 67 or an amino acid sequence that is up to 1, 2, 3, 4, 5, 6, 7, 8, 9, or 10 amino acids different from SEQ ID NO: 67.

10. The method of claim 2, wherein the AAV viral vector comprises an AAV capsid protein comprising or consisting of the amino acid sequence of SED ID NO: 3 or an amino acid sequence that is up to 2, 2, 3, 4, 5, 6, 7, 8, 9, or 10 amino acids different from SEQ ID NO: 3.

11. The method of any one of claims 2-3 and 10, wherein the AAV capsid protein comprises a VP3 portion comprising variable regions (VR) I to IX wherein:
(a) VR-II comprises amino acid sequence
(SEQ ID NO: 54)
DNNGVK,
(b) VR-III comprises amino acid sequence
(SEQ ID NO: 55)
NDGS,
(c) VR-IV comprises amino acid sequence
(SEQ ID NO: 56)
INGSGQNQQT
or
(SEQ ID NO: 171)
QSTGGTAGTQQ,
(d) VR-V comprises amino acid sequence
(SEQ ID NO: 57)
RVSTTTGQNNNSNFAWTA,
(e) VR-VI comprises amino acid sequence
(SEQ ID NO: 58)
HKEGEDRFFPLSG,
(f) VR-VII comprises amino acid sequence
(SEQ ID NO: 59)
KQNAARDNADYSDV,
(g) VR-VIII comprises amino acid sequence
(SEQ ID NO: 60)
ADNLQQQNTAPQI,
and
(h) VR-IX comprises amino acid sequence
(SEQ ID NO: 61)
NYYKSTSVDF.

12. The method of claim 11, wherein the VR-I region comprises SASTGAS (SEQ ID NO. 52), NSTSGGSS (SEQ ID NO. 53), SSTSGGSS (SEQ ID NO. 87), or NGTSGGST (SEQ ID NO: 170).

13. The method of any one of claims 1-11, wherein the ophthalmic disease or disorder is selected from the group consisting of dominant optic atrophy, retinitis pigmentosa, macular degeneration, an eye disorder related to mutations in the bestrophin-1 (BEST-1) gene, Leber congenital amaurosis, cone-rod dystrophy, Stargardt disease, choroideremia, Usher Syndrome, retinoschisis, Bietti's Crystalline Dystrophy and Achromatopsia.

14. The method of claim 13, wherein the retinitis pigmentosa is autosomal recessive, autosomal dominant, or X-linked.

15. The method of claim 13, wherein the eye disorder related to mutations in the BEST-1 gene is vitelliform macular dystrophy, age-related macular degeneration, autosomal dominant vitreoretinochoroidopathy, glaucoma, or cataract.

16. The method of any one of claims 1-15, wherein the AAV viral vector comprises an AAV vector genome encoding a gene selected from SPATA7, LRAT, TULP1, AIPL1, RPGR, AIPL1, ABCA4, CHM, MY07A, CDH23, USH2A, CLRN1, RS1, CYP4V2, CNGA3, CNGB3, GNAT2, RHO, PDE6B, PDE6C, PDE6H, OPA1, OPA3, and BEST-1.

17. The method of any one of claims 1-15, wherein the AAV viral vector comprises an AAV vector encoding an antisense RNA, microRNA, siRNA, or guide RNA (gRNA).

18. The method of any one of claims 1-17, wherein the ophthalmic disease or disorder is related to a dysfunction of optic nerve.

19. The method of any one of claims 1-17, wherein the ophthalmic disease or disorder is Dominant Optic Atrophy.

20. The method of claim 19, wherein the AAV viral vector comprises an AAV vector genome comprising an OPA1 or OPA3 transgene.

21. The method of any one of claims 1-17, wherein the ophthalmic disease or disorder is Retinoschisis.

22. The method of claim 21, wherein the AAV viral vector comprises an AAV vector genome comprising a RS1 transgene.

23. The method of any one of claims 1-22, wherein the para-retinal administration comprises injecting at a distance of between 0-13 millimeters (mm), between 0-10 mm, between 0-5 mm, or between 0-3 mm, from the surface of the retina in the posterior vitreous cavity of the eye.

24. The method of any one of claims 1-23, wherein the subject is a human.

25. A nucleic acid encoding an AAV capsid protein comprising a VP3 portion, wherein the VP3 portion comprises variable regions (VR) I to IX wherein:
(a) VR-II comprises amino acid sequence
(SEQ ID NO: 54)
DNNGVK,
(b) VR-III comprises amino acid sequence
(SEQ ID NO: 55)
NDGS,
(c) VR-IV comprises amino acid sequence
(SEQ ID NO: 171)
QSTGGTAGTQQ,
(d) VR-V comprises amino acid sequence
(SEQ ID NO: 57)
RVSTTTGQNNNSNFAWTA,
(e) VR-VI comprises amino acid sequence
(SEQ ID NO: 58)
HKEGEDRFFPLSG,
(f) VR-VII comprises amino acid sequence
(SEQ ID NO: 59)
KQNAARDNADYSDV,
(g) VR-VIII comprises amino acid sequence
(SEQ ID NO: 60)
ADNLQQQNTAPQI,
and
(h) VR-IX comprises amino acid sequence
(SEQ ID NO: 61)
NYYKSTSVDF.

26. The nucleic acid of claim 25, wherein the VR-I region comprises NGTSGGST (SEQ ID NO: 170).

27. The nucleic acid of claim 25, wherein the VP3 portion has the amino acid sequence of SEQ ID NO: 166.

28. The nucleic acid of any one of claims 25-27, wherein the AAV capsid protein further comprises i) a VP2 portion or ii) a VP1 portion and a VP2 portion.

29. The nucleic acid of any one of claims 25-27, wherein the encoded AAV capsid protein comprises an amino acid sequence at least 95%, at least 96%, at least 97%, at least 98%, at least 99%, or 100% identical to SEQ ID NO: 164 or an amino acid sequence having up to 1, 2, 3, 4, 5, 6, 7, 8, 9, or 10 amino acids different from SEQ ID NO: 164.

30. The nucleic acid of any one of claims 25-27, wherein the encoded AAV capsid protein comprises an amino acid sequence at least 95%, at least 96%, at least 97%, at least 98%, at least 99%, or 100% identical to SEQ ID NO: 165 or an amino acid sequence having up to 1, 2, 3, 4, 5, 6, 7, 8, 9, or 10 amino acids different from SEQ ID NO: 165.

31. The nucleic acid of any one of claims 25-27, wherein the encoded AAV capsid protein comprises an amino acid sequence at least 95%, at least 96%, at least 97%, at least 98%, at least 99%, or 100% identical to SEQ ID NO: 166 or an amino acid sequence having up to 1, 2, 3, 4, 5, 6, 7, 8, 9, or 10 amino acids different from SEQ ID NO: 166.

32. The nucleic acid of any one of claims 25-31, wherein the nucleic acid sequence is at least 95% identical to the nucleotide sequence selected from SEQ ID NOs: 167-169.

33. The nucleic acid of any one of claims 25-31, wherein the nucleic acid sequence is 100% identical to the nucleotide sequence selected from SEQ ID NOs: 167-169.

34. A vector comprising the nucleic acid of any one of claims 25-33.

35. An AAV capsid protein encoded by the nucleic acid of any one of claims 25-33.

36. The AAV capsid protein of claim 35, wherein the protein comprises the amino acid sequence of SEQ ID NO: 164, 165 or 166, or an amino acid sequence having up to 1, 2, 3, 4, 5, 6, 7, 8, 9, or 10 amino acids different from SEQ ID NO: 164, 165 or 166.

37. An AAV viral vector comprising the AAV capsid protein encoded by the nucleic acid of any one of claims 25-33 and an AAV vector genome, wherein the AAV vector genome comprises, in 5′ to 3′ orientation:
(a) a first AAV inverted terminal repeat,
(b) a promoter,
(c) a heterologous nucleic acid,
(d) a polyadenylation signal, and
(e) a second AAV inverted terminal repeat.

38. The AAV viral vector of claim 37, wherein the heterologous nucleic acid is operably linked to a constitutive promoter.

39. The AAV viral vector of claim 37 or 38, wherein the heterologous nucleic acid encodes a polypeptide.

40. The AAV viral vector of claim 37 or 38, wherein the heterologous nucleic acid encodes an antisense RNA, an siRNA, a microRNA, or a gRNA.

41. The AAV viral vector of any one of claims 37-41, wherein the AAV capsid protein comprises the amino acid sequence of SEQ ID NO: 164, 165 or 166.

42. An AAV viral vector comprising
(i) an AAV capsid protein having the amino acid sequence of SEQ ID NO: 164 and
(ii) an AAV vector genome, wherein the AAV vector genome comprises, in 5′ to 3′ orientation:
(a) a first AAV inverted terminal repeat,
(b) a promoter,
(c) a heterologous nucleic acid,
(d) a polyadenylation signal; and
(e) a second AAV inverted terminal repeat.

43. The AAV viral vector of any one of claims 37-39 and 41-42, wherein the heterologous nucleic acid encodes a polypeptide having at least 90% identity to any one of SEQ ID NOs: 142-144 and 177-181.

44. The AAV viral vector of claim 43, wherein the heterologous nucleic acid comprises a polynucleotide sequence having at least 90% identity to any one of SEQ ID NOs: 116-118 and 172-176.

45. A method of treating a disease or disorder comprising administering the AAV viral vector of any of claims 37-44 to a subject.

46. The method of claim 45, wherein the AAV viral vector is administered to the subject orally, rectally, transmucosally, inhalationally, transdermally, parenterally, intravenously, subcutaneously, intradermally, intramuscularly, intrapleurally, intracerebrally, intrathecally, intracerebrally, intraventricularly, intranasally, intra-aurally, intra-ocularly, peri-ocularly, topically, intralymphatically, intracistemally, intravitreally, para-retinally, or sub-retinally.

47. The method of claim 45 or 46, wherein the disease or disorder is amyotrophic lateral sclerosis (ALS), spinal muscular atrophy (SMA), Fabry disease, Pompe disease, CLN3 disease (or Juvenile Neuronal Ceroid Lipofuscinosis), recessive dystrophic epidermolysis bullosa (RDEB), juvenile Batten disease, autosomal dominant disorder, muscular dystrophy, hemophilia A, hemophilia B, multiple sclerosis, diabetes mellitus, Gaucher disease cancer, arthritis, muscle wasting, heart disease, intimal hyperplasia, epilepsy, Huntington's disease, Parkinson's disease, Alzheimer's disease, cystic fibrosis, thalassemia, Hurler's Syndrome, Sly syndrome, Scheie Syndrome, Hurler-Scheie Syndrome, Hunter's Syndrome, Sanfilippo Syndrome A (mucopolysaccharidosis IIIA or MPS IIIA), Sanfilippo Syndrome B (mucopolysaccharidosis IIIB or MPS IIIB), Sanfilippo Syndrome C, Sanfilippo Syndrome D, Morquio Syndrome, Maroteaux-Lamy Syndrome, Krabbe's disease, phenylketonuria, Batten's disease, spinal cerebral ataxia, LDL receptor deficiency, hyperammonemia, arthritis, macular degeneration, retinitis pigmentosa, ceroid lipofuscinosis, neuronal, 1 (CLN1), adenosine deaminase deficiency, Dominant Optic Atrophy, Retinoschisis, Stargardt disease, Bietti's Crystalline Dystrophy or BEST vitelliform macular dystrophy.

48. The method of claim 45 or 46, wherein the diseases or disorder is an ophthalmic disease or disorder.

49. The method of claim 48, wherein the ophthalmic disease or disorder is selected from the group consisting of dominant optic atrophy, retinitis pigmentosa, macular degeneration, an eye disorder related to mutations in the bestrophin-1 (BEST-1) gene, Leber congenital amaurosis, cone-rod dystrophy, Stargardt disease, choroideremia, Usher Syndrome, retinoschisis, Bietti's Crystalline Dystrophy and Achromatopsia.

50. The method of any of claims 45-49, wherein the subject is a human.
